(19) United States
(12) Patent Application Publication
(10) Pub. No.: US 2025/0111907 A1
(43) Pub. Date: Apr. 3, 2025

(54) PERSONALIZED TREATMENT RECOMMENDATION SYSTEM

(71) Applicant: The Regents of the University of California, Oakland, CA (US)

(72) Inventors: Vivek Rudrapatna, San Francisco, CA (US); Vignesh Ravindranath, San Francisco, CA (US); Shan Wang, San Francisco, CA (US)

(21) Appl. No.: 18/899,323

(22) Filed: Sep. 27, 2024

Related U.S. Application Data
(60) Provisional application No. 63/541,407, filed on Sep. 29, 2023.

(51) Int. Cl. G16H 10/20; G16H 50/20; G16H 50/70

(52) U.S. Cl. CPC G16H 10/20; G16H 50/20; G16H 50/70

(57) ABSTRACT

This invention provides such new and useful methods and systems for accurately comparing treatment efficacy and safety using heterogeneous clinical trials in the absence of a common control group. To accomplish this, the invention leverages both novel regression techniques and novel simulation techniques to normalize heterogeneous clinical trials to a common background, and to analytically isolate the portion of the patient response specifically attributable to a given treatment and not the placebo effect. The sequential regression and simulation techniques of the disclosed methods and systems further allow for the outcomes of subjects of heterogeneous clinical trials to be simulated as a function of treatment and patient-level features, enabling the creation of decision support tools capable of making personalized treatment recommendations.

### CROSS-REFERENCE TO RELATED APPLICATION

Pursuant to 35 U.S.C. § 119 (e), this application claims priority to the filing date of U.S. Provisional patent application Ser. No. 63/541,407 filed Sep. 29, 2023, the disclosure of which is herein incorporated by reference in its entirety.

### STATEMENT REGARDING FEDERALLY SPONSORED RESEARCH

This invention was made with government support under grant nos. TR001872, T32DK007007, and K99LM014099, awarded by the National Institutes of Health (NIH). The government has certain rights in the invention.

### INTRODUCTION

Although head-to-head trials are the gold-standard for comparing medical treatments, they are often expensive and infeasible. Fortunately, the advent of clinical trial data sharing platforms has created new opportunities for making discoveries and answering important questions using previously collected data. Thus, network meta-analyses (NMAs) have become the primary source of evidence on comparative efficacy and safety for the treatment of several diseases including, e.g., Crohn's disease (CD). These studies utilize summary statistics from multiple trials as the basis for inferring the relative effectiveness of treatments.

While NMAs have provided important information about relative treatment effects, they are subject to many limitations. They assume that the included trials are homogenous across multiple dimensions (e.g., cohort risk profiles, study procedures, placebo effects), and that each included trial represents a random sample of the potential comparisons of interest (e.g., an equal chance that an included trial will compare drug A to B, rather than comparing drug A to placebo). Additionally, another key assumption NMAs make in justifying the application of their results to practice is that the cohorts under study are an unbiased sample of real-world populations having a given disease or condition of interest. Furthermore, these models ignore the potential role of patient-level variation in explaining treatment outcomes. As such, although NMAs are able to infer relative treatment effects by averaging over the outcomes of a potentially heterogeneous population, they lack utility in identifying patient subgroups whose responses deviate from the majority.

### SUMMARY

Thus, there is a need for improved and useful methods and systems for accurately comparing treatment efficacy and safety using heterogeneous clinical trials in the absence of a common control group. This invention provides such new and useful methods and systems, addressing the limitations mentioned above. To accomplish this, the invention leverages both novel regression techniques, and novel simulation techniques, to normalize heterogeneous clinical trials to a common background and to analytically isolate the portion of the patient response specifically attributable to a given treatment, and not the placebo effect. The sequential regression and simulation techniques of the disclosed methods and systems further allow for the outcomes of subjects of heterogeneous clinical trials to be simulated as a function of treatment and patient-level features, enabling the creation of a decision support tool capable of making personalized treatment recommendations.

In one aspect, methods for modeling the placebo response of subjects of a plurality of clinical trials, regarding the treatment of a disease or condition with a medical intervention, separately from the medical intervention attributable component of the subject response using patient-level features of the subjects are provided. Aspects of the methods include: obtaining individual participant data from a plurality of clinical trials regarding the treatment of the disease or condition with a medical intervention, wherein the clinical trials lack a shared control group; selecting one or more patient level features and an outcome variable enabled by the individual participant data; filtering and harmonizing a subset of the original patient data including only data associated with individuals that have received a placebo; and fitting a regression model to the filtered and harmonized subset of data using the one or more patient level features of each individual.

In certain embodiments, the placebo regression model is a mixed effects regression model. In some embodiments, the mixed effects placebo regression model comprises a statistical regression model such as, e.g., a linear mixed effects regression model. In some cases, the mixed effects placebo regression model comprises a machine learning (ML) model such as, e.g., a supervised ML model. In these instances, the supervised ML model may comprise a tree-based model, a Bayesian model, a neural network (NN), and/or Gaussian process model. In some embodiments, the one or more patient-level features of each individual are modeled as fixed effects and the clinical trial associated with each individual is modeled as a random effect. In some embodiments, the year each clinical trial was performed is modeled as a fixed effect. In some embodiments, the one or more patient-level features include demographic features, modifiers of treatment response, proxies of chronic disease burden, and/or proxies of treatment response.

In certain embodiments, the fitted placebo regression model is used to identify relationships between the outcome variable and the one or more patient level features. In some embodiments, the fitted placebo regression model is used to improve the design and statistical power of clinical trials for a disease or condition. In some embodiments, the fitted placebo regression model is used to improve the robustness of external control arm studies such as, e.g., single-arm intervention studies. In some embodiments, the fitted placebo regression model is used to model the response of subjects having a disease or condition to a medical intervention, e.g., as discussed in greater detail below.

In another aspect, methods of isolating the portion of each subject's response that is attributable to a medical intervention using a placebo regression model and patient-level features of the subjects, for subjects of a plurality of clinical trials having a disease or condition, in order to model the effect of the medical intervention on subjects having the disease or condition are provided. Aspects of the methods include: obtaining individual participant data from a plurality of clinical trials regarding the treatment of the disease or condition with one or more medical interventions, wherein the clinical trials lack a shared control group; selecting one or more patient level features and an outcome variable enabled by the individual participant data; filtering and harmonizing a first subset of the individual patient data including only data associated with individuals that have received a placebo and a second subset of the individual patient data including only data associated with individuals that have received a first medical intervention; fitting a placebo regression model to the filtered and harmonized first subset of individual participant data in order to determine an effect of each patient level feature on the outcome variable; determining a component of the outcome variable attributable to the first medical intervention for each individual of the second subset using the one or more patient level features of each individual and the fitted placebo regression model; fitting a medical intervention regression model to the first medical intervention attributable component and the one or more patient level features of each individual of the second subset. In some embodiments, the fitted medical intervention model is used to identify relationships between the first medical intervention attributable component and the one or more patient level features.

In certain embodiments, the medical intervention model is a mixed effects regression model. In some embodiments, the mixed effects intervention regression model comprises a statistical regression model such as, e.g., a linear mixed effects regression model. In some cases, the mixed effects intervention regression model comprises a machine learning (ML) model such as, e.g., a supervised ML model. In these instances, the supervised ML model may comprise a tree-based model, a Bayesian model, a neural network (NN), and/or Gaussian process model. In some embodiments, the one or more patient-level features of each individual are modeled as fixed effects and the clinical trial associated with each individual is modeled as a random effect. In some embodiments, the one or more patient-level features include demographic features, modifiers of treatment response, proxies of chronic disease burden, and/or proxies of treatment response.

In certain embodiments, the individual participant data is obtained from a plurality of clinical trials regarding the treatment of the disease or condition with two or more different medical interventions. In some embodiments, the methods further include: filtering and harmonizing a third subset of the individual patient data including only data associated with individuals that have received a second medical intervention; determining a component of the outcome variable attributable to the second medical intervention for each individual of the third subset using the one or more patient level features of each individual and a fitted placebo regression model; fitting a second medical intervention regression model to the second medical intervention attributable component and the one or more patient level features of each individual of the third subset. In some embodiments, the methods further include ranking the first and second medical interventions for an individual based on the one or more patient level features of the individual and each fitted medical intervention model.

In certain embodiments, the fitted medical intervention regression models are used to identify subgroups of individuals having different preferential responses to different medical interventions. In some embodiments, the fitted medical intervention regression models are used to identify selection bias of individuals into clinical trials regarding the treatment of a disease or condition. In some embodiments, the fitted medical intervention regression models are used to design a clinical trial testing the efficacy and safety of one or more medical interventions. In some embodiments, the fitted medical intervention regression models are used to improve the design and statistical power of clinical trials for a disease or condition. In some embodiments, the fitted medical intervention regression models are used to create a decision support tool providing personalized treatment recommendations to clinicians selecting treatments for patients having a disease or condition.

Also provided are systems for performing the methods described herein as well as computer products and non-transitory computer readable storage media including the instructions of the memory of the systems described herein.

### BRIEF DESCRIPTION OF THE FIGURES

FIG. 1 depicts an overview of a study performed using modeling methods in accordance with an embodiment of the invention. (a). Clinical trials were found using clinicaltrials.gov and sought for retrieval on the YODA and Vivli platforms. Individual participant data (IPD) from trials that collected CDAI scores at week 8 visits were then aggregated and harmonized. (b). Two linear mixed effect models-placebo-attributable and ADA-attributable-were developed from the harmonized data to partition the CDAI reduction based on baseline covariates (age, sex, BMI, etc.). Disease activity reduction was partitioned into placebo attributable (square) and drug-attributable (circle) effects. IPD (solid lines) were used to predict or simulate data (dashed lines). (c). Using the adalimumab (ADA) attributable model, the outcomes of the placebo group from the ustekinumab (UST) trials were simulated under a counterfactual scenario where they had instead been assigned to receive ADA. (d). Results from a simulated head-to-head trial were compared against a recently completed head-to-head trial, SEAVUE, to externally validate the proposed method. ADA=adalimumab, CZP=certolizumab pegol, NTX=natalizumab, UST=ustekinumab.

FIG. 2 illustrates a summary of randomized controlled trial study designs. Data harmonization required careful understanding of the study designs. All treatment arms that involved 8 weeks of consistent exposure to either placebo or (blue) or active treatment at the FDA-approved doses (red) were included. R=randomized and blinded; O=open label.

FIG. 3 provides a directed acyclic graph of the modeling strategy. (a). A directed acyclic graph (DAG) of the drug attributable effect. In addition to the active treatment itself, patient demographics (age, sex, BMI), baseline Crohn's disease activity (baseline CDAI, CRP, location), and treatment history (prior use of TNFis, current use of oral corticosteroids and immunomodulators) are all modelled as contributing to the drug attributable effect. The non-drug covariates are effect modifiers and are implicitly modeled as two-way interaction terms with the active drug. (b). A DAG of the drug independent effect (i.e., placebo effect). The same covariates except for the treatment term are modeled as effect modifiers and are implicitly represented as two-way interactions with the placebo effect. (c). Drug attributable and drug independent effects have additive effects on the overall clinical remission at week 8 (CDAI<150), with any individual trial reflecting a noisy measurement of the true effect due to unmodeled heterogeneity in study design and execution (random effect). TNFi=tumor necrosis factor inhibitor.

FIG. 4 provides a table characterizing the baseline covariates of included studies and simulated head-to-head trial. Placebo arms from the CLASSIC, EXTEND, and NCT02499783 studies were not included due to the absence of an 8-week parallel arm placebo group (see FIG. 2). CRP=c-reactive protein, TNFi=tumor necrosis factor inhibitor.

FIG. 5 provides a table of the mixed effect linear regression outputs for the placebo attributable (n=1310) and ADA attributable (n=239) models in accordance with an embodiment of the invention. For training, Year was centered by subtracting 2000, Baseline CDAI was centered by subtracting 300, Age was centered by subtracting 35, BMI was centered by subtracting 20, and CRP (mg/L) was centered by subtracting 10. The placebo attributable model (intraclass correlation coefficient 0.02) trial random intercepts were found to be −12.808 (PRECISE1), −7.975 (UNITI1), −6.328 (CERTIFI), 6.077 (ENACT), 8.669 (ENCORE), and 12.366 (UNITI2). The ADA attributable model (intraclass correlation coefficient 0.05) trial random intercepts were found to be −20.215 (CLASSIC), 9.439 (EXTEND), and 10.775 (NCT02499783).

FIG. 6 provides a table comparing clinical remission rates at week 8 for the TNF-naive ustekinumab (UST) cohort and TNF-naive adalimumab (ADA) cohort for the SEAVUE study, the primary analysis (simulation of SEAVUE), sensitivity analyses, and negative control. Because missing week 8 CDAI values were highest for trials PRECISE1 and ENACT, their participant-level data was removed (N=1482) from the first sensitivity analysis to account for potential bias. In the complete case sensitivity analysis, all participants with missing week 8 CDAI values (N=361) were removed. In the information leakage sensitivity analysis, participants from an ustekinumab study (N=1191) were removed from training the placebo-attributable model to avoid potential information leakage when simulating the adalimumab (ADA) arm (FIG. 1C). The negative control summarizes the clinical remission rates at week 8 for TNF-naive participants from the adalimumab studies without applying the regression-based correction method in accordance with an embodiment of the invention. The final column corresponds to the results of null hypothesis testing, that of no statistically significant difference between each simulated result and the published SEAVUE results.

FIG. 7 depicts a flow diagram illustrating the selection of studies. *=some studies met more than one criterion. †=15 studies were retrieved and consolidated on the Vivli platform; however, only 9 studies were used for analysis as these studies captured CDAI measurement at week 8 and could be compared with the SEAVUE study.

FIG. 8 illustrates Cochrane's risk-of-bias tool for randomized trials version 2 (ROB2). Green, yellow, and red indicate low, moderate, and high risk of bias respectively.

FIGS. 9A to 9B illustrate the reproducibility of the obtained published data. (A). Plots of aggregated data versus published data for baseline covariates and outcome variables as a measure of quality control. Each dot represents the mean variable estimate for a given study treatment group (placebo, active). Data were not displayed if the study did not report the variable mean in its original article. Upper and lower lines in plots correspond to ±10% error bounds. (B). The percentage of missing covariates by study. Approximately 0.2% of BMI values, 2% of CRP values, and 11% of week 8 CDAI values were missing after data harmonization and required imputation. Median imputation by study was used to impute missing BMI and CRP values. Last observation carried forward (LOCF) was used to impute missing week 8 CDAI values; CDAI observations from week 6, week 4, week 3, or week 2 were candidates for LOCF.

FIGS. 10A to 10B depict the results of a leave-one-trial-out analysis. (A). A leave-one-trial-out analysis, where blue and red dots represent the true and predicted mean CDAI reduction respectively. (B). Q-Q plot of the model residuals (difference in true and predicted mean CDAI reduction values per study) to assess residual normality (p=0.4 by the Shapiro-Wilk test).

FIGS. 11A to 11D provide plots of the placebo-attributable model residuals to visually check linear regression model assumptions. (A). Plot of the model residuals versus index to assess residual independence. Dotted lines represent ±2σ. (B). Plot of the model residuals versus the fitted values to assess residual homoscedasticity. (C). Q-Q plot of the model residuals to assess residual normality. (D). Plot of the model residuals versus each continuous covariate to assess linearity between covariates and the outcome variable.

FIG. 12 depicts the major inclusion/exclusion criteria of included studies of Example 1. If the trial protocol was not publicly available, and if the corresponding manuscript or clinical study report was silent on a given criteria, the field was annotated as NA. If the trial protocol was available and if it was clear that a given criterion was not applied for cohort selection, the field was annotated with an X. In many trials (e.g., ENACT, ENCORE), a history of TNF-naive or intolerance/failure was not a requirement and was captured as a participant-specific covariate for regression-based control. In other scenarios, the trial-specific covariate implicitly applied to all trial participants (e.g., TNF-naive status in PRECISE1). Trials were generally consistent on the target patients of study in terms of inclusion and exclusion criteria. To address the possibility of residual heterogeneity due to the lack of perfectly consistent eligibility criteria or unmeasured covariates, a trial-specific random effect was included in the final regression models. *=either absence of exposure or absence of prior intolerance or inadequate response.

FIG. 13 provides a table of mean 5-fold cross-validation (CV) root mean squared error (RMSE) scores of various predictive models. All models were tested against the same stratified 5-fold datasets to ensure results were comparable. RMSE scores reflect the model's ability to predict CDAI reduction at week 8 due to a placebo treatment given baseline covariates (age, sex, BMI, etc.). The stacked ensemble model was built from 9 default base models using the mljar AutoML package.

FIG. 14 depicts an overview of an analysis performed using modeling methods in accordance with an embodiment of the invention. (a). Clinical trials were found using clinicaltrials.gov and sought for retrieval on the YODA and Vivli platforms. Individual participant data (IPD) from trials that collected CDAI scores at week 6 visits were then aggregated and harmonized. (b). Using sequential regression and simulation, a method for normalizing clinical trial data against a common placebo rate in accordance with an embodiment of the invention, a placebo-attributable model and three drug-attributable models-anti-integrin, anti-interleukin-12/23, and anti-TNF—were developed. Disease activity reduction was partitioned into placebo attributable (square), and drug-attributable (circle) effects based on baseline covariates (age, sex, BMI, etc.). IPD (solid lines) were used to predict or simulate data (dashed lines). (c). The drug-attributable models were utilized to simulate patient-level outcomes post-treatment (counterfactuals). Pairwise t-tests (p<0.05) were conducted to compare and rank the mean responses for all drug classes-anti-integrin vs anti-interleukin-12/23, anti-integrin vs anti-TNF, and anti-interleukin-12/23 vs anti-TNF—and assign patients into one of seven subgroup memberships (see FIG. 23). (d). Lastly, the models were re-packaged into a prototype decision support tool that uses manual inputs and optionally, OMOP-formatted data, to recommend treatments for individual patients.

FIG. 15 provides a detailed comparison of three major subgroup cohorts found in the trial-based cohort (N=5703): prefer anti-TNF only (N=2061, red), prefer anti-TNF or anti-IL-12/23 (N=355, blue), and prefer anti-IL-12/23 only (N=139, green). The bar plots in the upper graphs show the average placebo (P) and drug-class (D) attributable effects for each subgroup. Superior drug classes (left of bolded vertical line) reduce disease activity (CDAI reduction) by 30-40 points more on average compared to non-superior drug-classes (right of bolded vertical line). The middle and bottom plots compare the proportions and distributions of covariates for each subgroup.

FIG. 16 illustrates an example of a user interface and output of an R Shiny treatment recommendation dashboard with inputs in accordance with an embodiment of the invention.

FIG. 17 depicts a PRISMA flow diagram illustrating selection of studies. *=some studies met more than one criterion. †=all 15 studies were retrieved and consolidated on the Vivli platform; however, only 9 studies were used for analysis as these studies captured CDAI measurement at week 8 and could be compared with the SEA VUE study.

FIG. 18 illustrates Cochrane's risk-of-bias tool for randomized trials version 2 (ROB2). Green, yellow, and red indicate low, moderate, and high risk of bias respectively.

FIG. 19 illustrates summaries of different study designs. Data harmonization required careful understanding of the study designs. All treatment arms that involved 6 weeks of consistent exposure to either placebo or (blue) or active treatment at the FDA-approved doses (red) were included. R=randomized and blinded; O=open label.

FIG. 20 depicts a flow chart of real-world patients in the University of California Health (UCH) Data Warehouse that correspond to the anti-IL-12/23 subgroup (approximately women over 50 years of age (see FIG. 2)). Of the 22,670 patients diagnosed with Crohn's disease in the database, 5647 (25%) are women over the age of 50. 4,250 of those women received their first biologic after 2016, when all biologics in the analysis were open on the market. Of those, only 1,052 (25%) women received an anti-IL-12/23 as their first line biologic. Query was run on Apr. 5, 2023.

FIG. 21 depicts a table providing the baseline characteristics of the study cohort used for meta-analysis in accordance with an embodiment of the invention, stratified by trial. Placebo arms from ACCENT, CLASSIC, EXTEND, NCT02499783, PRECISE2, and SONIC studies were not included due to the absence of a 6-week parallel arm placebo group (see FIG. 17). CRP=c-reactive protein, TNF=tumor necrosis factor.

FIG. 22 provides a table of outputs of linear mixed effect regression models for various Crohn's Disease treatments in accordance with an embodiment of the invention. A total of four linear mixed effects regression models were fit: one placebo model and three nested models of the drug class-attributable response. Rows correspond to the fixed effect parameters of each model, and columns correspond to the estimated coefficients, standard errors, and Wald test p-values with bolding corresponding to significance at the 0.05 level. Year was not used for the drug class models due to insufficient variation (few trials per drug class, clustered together in calendar time).

FIG. 23 depicts a table of treatment subgroups. The finalized mixed effects models were used to simulate counterfactual outcomes (e.g., mean response, residual uncertainty) for every patient under all possible treatment scenarios. The modeled outcomes and the associated uncertainties in these outcomes were used to perform pairwise t-testing to assess evidence for rank-ordered preferences across drug classes. Distinct patterns of rank-orderings were used to establish membership in one of 6 subgroups. Subjects without sufficient statistical evidence (alpha=0.05) of a more efficacious response to any one drug classes were placed into a 7^{th} category (no preference). TNF=anti-tumor necrosis factor, IL=anti-interleukin-12/23, INT=anti-integrin.

FIG. 24 depicts the major inclusion/exclusion criteria of included studies of Example 2. If the trial protocol was not publicly available, and if the corresponding manuscript or clinical study report was silent on a given criteria, the field was annotated as NA. If the trial protocol was available and if it was clear that a given criterion was not applied for cohort selection, the field was annotated with an X. In many trials (e.g., ENACT), a history of TNF-naive or intolerance/failure was not a requirement and was captured as a participant-specific covariate for regression-based control. In other scenarios, the trial-specific covariate implicitly applied to all trial participants (e.g., TNF-naive status in PRECISE1). Trials were generally consistent on the target patients of study in terms of inclusion and exclusion criteria. To address the possibility of residual heterogeneity due to the lack of perfectly consistent eligibility criteria or unmeasured covariates, a trial-specific random effect was included in the final regression models. TNFi=tumor necrosis factor inhibitor. *=either absence of exposure or absence of prior intolerance or inadequate response.

FIG. 25 provides a table of baseline characteristics of the anti-interleukin-12/23 subgroup, stratified by gender (N=139).

FIG. 26 provides a table of the results of a simulation in accordance with an embodiment of the invention. A power analysis was conducted to evaluate the number of participants required per trial arm (100, 250, 500) for a hypothetical head-to-head randomized controlled trial comparing anti-IL-12/23 drugs against anti-TNF drugs for three cohorts of patients over 50 years old: men and women (cohort 1), women only (cohort 2), and men only (cohort 3). The results correspond to the proportion of trials where the anti-IL-12/23 arm significantly outperformed the anti-TNF arm. Over 1000 simulations were performed for each pair of cohort and arm size.

FIG. 27 provides a flow diagram depicting a method for providing personalized treatment recommendations for a subject in accordance with an embodiment of the invention.

FIG. 28 depicts a table providing the results of a 10-fold cross-validation analysis demonstrating the robustness of treatment subgroup allocation using mixed effect regression models in accordance with an embodiment of the invention.

FIG. 29 depicts a table providing the results of a 10-fold cross-validation analysis demonstrating the consistent finding of an anti-interleukin-12/23 preference subgroup (female over 50) using mixed effect regression models in accordance with an embodiment of the invention.

### DETAILED DESCRIPTION

The present disclosure provides methods and systems for accurately comparing treatment efficacy and safety using heterogeneous clinical trials in the absence of a common control group. Aspects of the methods include: obtaining individual participant data from a plurality of clinical trials regarding the treatment of a disease or condition with a medical intervention, wherein the clinical trials lack a shared control group; selecting one or more patient level features and an outcome variable enabled by the individual participant data; filtering and harmonizing a subset of the original patient data including only data associated with individuals that have received a placebo; and fitting a regression model to the filtered and harmonized subset of data using the one or more patient level features of each individual. Aspects of the present invention further include: filtering and harmonizing a second subset of the individual patient data including only data associated with individuals that have received a first medical intervention; determining a component of the outcome variable attributable to the first medical intervention for each individual of the second subset using the one or more patient level features of each individual and the fitted placebo regression model; and fitting a medical intervention regression model to the first medical intervention attributable component and the one or more patient level features of each individual of the second subset. Also provided are systems for performing the methods described herein as well as non-transitory computer readable storage media and computer products.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods for accurately comparing treatment efficacy and safety, using heterogeneous clinical trials in the absence of a common control group, are provided. Aspects of the methods include: obtaining individual participant data from a plurality of clinical trials regarding the treatment of a disease or condition with a medical intervention, wherein the clinical trials lack a shared control group; selecting one or more patient level features and an outcome variable enabled by the individual participant data; processing a subset of the original patient data including only data associated with individuals that have received a placebo; and fitting a regression model to the processed subset of data using the one or more patient level features of each individual. Aspects of the present invention further include: processing a second subset of the individual patient data including only data associated with individuals that have received a first medical intervention; determining a component of the outcome variable attributable to the first medical intervention for each individual of the second subset using the one or more patient level features of each individual and the fitted placebo regression model; and fitting a medical intervention regression model to the first medical intervention attributable component and the one or more patient level features of each individual of the second subset.

As described above, embodiments of the methods include obtaining individual participant data (IPD) from a plurality of clinical trials regarding the treatment of a disease or condition with a medical intervention. By obtain is meant to make the IPD accessible or available for the subsequent steps of the methods (e.g., processing, fitting a placebo regression model, etc.). By IPD is meant data collected or recorded for each individual participant in a research study or clinical trial (i.e., data specific to each individual participant). The IPD may be obtained through any available means, and from any available source. In some embodiments, IPD may include electronic IPD or, i.e., IPD existing in a digital form. Electronic IPD may include structured data (i.e., data stored in a predefined structured format, e.g., for ease of sorting, searching and analysis) and/or unstructured data. In some embodiments, the IPD may be obtained by gaining access to a database (e.g., a data-sharing platform) storing IPD from a large number of different clinical trials.

The terms “subject”, “individual”, “patient”, and “participant” are used interchangeably herein and may refer, e.g., to a subject participating in a clinical trial (e.g., a subject for which IPD is obtained) or a subject for which a specific medical intervention is recommended (e.g., a subject for which electronic health record data (EHR) is obtained) depending on context. The subject is preferably human, e.g., a child, an adolescent, or an adult (such as a young, middle-aged, or elderly adult) human. In some cases, the subject is sixty years of age or older. In other cases, the subject is younger than sixty years of age.

In some embodiments, the IPD (e.g., digitally accessible IPD) may be obtained by transmitting the data from a database including (e.g., storing, or providing access or guidance to) a large number of different clinical trials. In some cases, the database may include fifty clinical trials or more, such as one hundred clinical trials or more, or five hundred or more, or one thousand or more, or three thousand or more, or five thousand or more, or ten thousand or more. Transmitting can include any manner of sending, passing, or conveying the IPD from the database to a means for performing a subsequent step or steps of the methods (e.g., a processor, computer program or application, lines of computer code, etc.). In some instances, the IPD may be obtained, at least in part, by converting the data to a form compatible with a subsequent step or steps of the methods. In some embodiments, the IPD may be converted from a format difficult for machines to interpret to a format in a standard computer language that can be read automatically by a machine. In some cases, optical character recognition (OCR) software may be used to convert IPD to a form compatible with a subsequent step or steps of the methods. For example, in cases where IPD is stored in an image format (e.g., a PDF or JPEG format), the IPD may be converted to a JSON format, an XML format, a CSV format, a CSON format, an HTML format, etc. In these cases, organizational or categorical information structuring or classifying the IPD may be manually entered. In some cases, organizational or categorical information may be automatically identified from IPD using, e.g., lines of computer code and rules-based approaches. In some instances, the IPD may be obtained by scanning or imaging a plurality of clinical trial or clinical study documents existing in hard copy form, followed optionally by conversion of the resulting image files in any of the manners discussed above.

In embodiments where IPD is obtained from a database including (e.g., storing, or providing access or guidance to) a large number of different clinical trials, the database may be aggregated or generated by any entity, including, but not limited to, a medical center, a nonprofit organization, an insurance provider, a government organization, an academic institution, a pharmaceutical or medical corporation, etc. In some embodiments, the IPD is obtained from the Vivli data-sharing platform, the Yale University Open Data Access (YODA) project, ClinicalTrials.gov, the Cochrane Library, the WHO International Clinical Trials Registry Platform (ICTRP), the European Union Clinical Trials Database, etc.

The IPD may include the data of individuals of any demographic or cohort. For example, the IPD may include data of individuals of any sex, gender, age, ethnicity, or race. In some cases, the IPD may include the data of individuals associated with a population or cohort of interest. By population or cohort of interest is meant a group of people banded together or treated as a group, such as a specific demographic of individuals. For example, the cohort of interest may be individuals experiencing or affected by (e.g., at risk for) a specific disease or condition. In these instance, the IPD may be obtained from clinical trials associated with the specific disease or condition such as, e.g., clinical trials associated with treating or addressing the disease or condition. In these cases, the disease or condition may be any disease or condition that impairs or affects the normal functioning of the body. In some instances, the disease or condition may be, e.g., an infectious disease, deficiency disease, hereditary disease, or physiological disease.

In embodiments where the disease or condition is an infectious disease, the infectious disease may be, e.g., a bacterial disease or infection (such as, e.g., syphilis, pneumonia, tetanus, and/or tuberculosis), a viral disease or infection (such as, e.g., chickenpox, measles, herpes, the common cold, or COVID-19), a fungal disease or infection (such as, e.g., ringworm infection, athlete's foot, or yeast infections), or a parasite or parasitic disease (such as, e.g., malaria).

In embodiments where the disease or condition is a deficiency disease, the deficiency disease may be, e.g., malnutrition, scurvy, rickets, osteoporosis, or a birth defect. In embodiments where the disease or condition is a hereditary disease, the hereditary disease may be, e.g., cystic fibrosis, Huntington's Disease, sickle cell anemia, acute hepatic porphyria, primary hyperoxaluria, hereditary transthyretin amyloidosis, a birth defect, etc. In some cases, the disease or condition may be affected by, but not unilaterally caused by, genetics or may be a polygenic disease. In these instances, the disease or condition may be caused by a combination of genetic and environmental factors and may be asthma, an autoimmune disease such as multiple sclerosis, cancer, ciliopathy, cleft palate, diabetes, heart disease, hypertension, an inflammatory bowel disease (e.g., Crohn's disease), an intellectual disability, a mood disorder, obesity, refractive error, infertility, schizophrenia, or any number of a variety of mental disorders. In embodiments where the disease or condition is a physiological disease, the physiological disease may be, e.g., diabetes, cancer, hypertension, or heart disease. In some cases, the disease or condition may include any disease or condition caused by environmental factors, behavior, or diet.

In some cases, the disease or condition may psychological disease or condition such as, e.g., an anxiety disorder, depression, bipolar disorder, post-traumatic stress disorder (PTSD), schizophrenia, an eating disorder, a disruptive behavior and/or dissocial disorder, or a neurodevelopmental disorder. In some instances, the disease or condition may be hypothermia, hyperthermia, or toxin exposure or may result from exposure to prolonged or extreme hot or cold temperatures. In some cases, the disease or condition may result from an injury or may affect mobility. For example, the disease or condition may include, but is not limited to, a burn, cuts or scrapes, internal bleeding, traumatic injuries, arthritis, tendonitis, a tendon or myotendinous tear, broken or fractured bones, a hernia, old age, paralysis, chronic health problems such as, e.g., chronic pain or chronic problems associated with bad posture, etc.

In some instances, the IPD may include, or consist of, data regarding individuals diagnosed with a specific disease or condition. In other cases, the IPD may be associated with individuals at risk for a specific disease or condition (e.g., pregnancy, HIV, influenza, male androgenetic alopecia, etc.). In some instances, the IPD may include, or consist of, data regarding individuals that have been administered or exposed to a medical intervention in order to address the specific disease or condition experienced by or affecting the individuals. In some cases, IPD may be obtained from clinical trials associated with addressing or treating the specific disease or condition with the medical intervention. In some instances, the IPD pertains to individuals exposed to or receiving the medical intervention (i.e., the IPD is associated with individuals of a treatment group). In some instances, the IPD may pertain to individuals diagnosed with or experiencing a specific disease or condition that may be addressed or treated by the medical intervention, but have not received or been exposed to the medical intervention (i.e., the IPD is associated with individuals of a placebo group). In some instances, the IPD pertains to individuals that have received different medical interventions for the same disease or condition.

As described above, embodiments of the methods include obtaining IPD from a plurality of clinical trials regarding the treatment of the disease or condition with one or more medical interventions, wherein the clinical trials lack a shared control group. By control group is meant participants of a clinical who do not receive a medical intervention, and instead serve as a comparison group for participants of the clinical trial who have received the medical intervention. In some cases, the control group is matched as closely as possible to the experimental group (i.e., the group of clinical trial participants who received the medical intervention) including, e.g., in age, gender, social class, ethnicity, etc. By medical intervention is meant a treatment, procedure, or other action taken to prevent or treat disease, or improve health in any number of a variety of ways. In some embodiments, the medical intervention may include the administration of a pharmaceutical composition, medical device, surgery, and/or therapy to a subject or an alteration of lifestyle (e.g., responsibilities of employment, diet, exercise plans, etc.) by a subject.

In some embodiments, the medical intervention may include the administration of a pharmaceutical composition to a subject. In these instances, the pharmaceutical composition may include any dosage form configured to deliver an active pharmaceutical ingredient (API) of a pharmaceutical composition to a subject. The dosage forms may vary depending on the desired route of administration of a pharmaceutical composition and API therein. By route of administration is meant the way an API enters into an individual's system (e.g., how an API is taken into an individual's body). For example, routes of administration may include, but are not limited to, administrating a pharmaceutical composition and API therein orally, sublingually, topically, transdermally, rectally, vaginally, nasally, optically, by inhalation, and by injection. The pharmaceutical composition may include any number of a variety of APIs that are provided to treat or address (e.g., prevent) any number of a variety of diseases or conditions, such as any of the diseases or conditions described above. These API's include, without limitation, opiates, drugs used in psychiatry or in the treatment of schizophrenia, drugs used for birth control, cytotoxic substances, analgesics, anti-inflammatories (e.g., anti-TNF, anti-integrins, or anti-IL-12/23), antipyretics, antibodies, antibiotics, antimicrobials, anxiolytics, laxatives, anorexics, antihistamines, antidepressants, anti-asthmatics, antidiuretics, anti-flatulents, antimigraine agents, antispasmodics, sedatives, steroids, anti-hyperactives, antihypertensives, tranquilizers, decongestants, beta blockers, peptides, proteins, genes and vectors used for gene therapy, oligonucleotides and other substances of biological origin, biologically active organic compounds, and combinations thereof. In some instances, the pharmaceutical composition may include a vitamin, mineral and/or dietary supplement.

In some embodiments, the medical intervention may include the application of a medical device to a subject. In these instances, the medical device may include, but is not limited to, medical devices provided to enhance mobility or communication, regulate biological function, measure or track physiological data, image, correct a biological deficiency or dysmorphic feature, and/or administer a pharmaceutical composition (e.g., as described above). In embodiments where the medical device is provided to enhance mobility or communication, the medical device may be, e.g., a prosthetic (e.g., a prosthetic limb, organ, or a neuroprosthetic such as a pacemaker or cochlear implant), an artificial hip or joint, a physical therapy machine, plates or screws, etc. In embodiments where the medical device is provided to regulate biological function, the medical device may be, e.g., a medical ventilator, incubator, anesthetic machine, heart-lung machine, ECMO, dialysis machine, IUD, etc. In embodiments where the medical device is provided to measure or track physiological data, the medical device may be, e.g., a wearable device (e.g., a smartwatch or a wearable sensor) or an implanted medical device. In embodiments where the medical device is provided to image, the medical device may be, e.g., an ultrasound and/or MRI machine, PET scanner, x-ray machine, etc. In embodiments where the medical device is provided to correct a biological deficiency or dysmorphic feature, the medical device may be, e.g., a medical laser or a surgical machine (e.g., a LASIK surgical machine). In embodiments where the medical device is provided to administer a pharmaceutical composition, the medical device may be, e.g., an insulin pump or a hormonal IUD.

In some embodiments, the medical intervention may include the application of a surgery or therapy to a subject. In these instances, the medical intervention may include, but is not limited to, surgeries and/or therapies provided to treat or address (e.g., prevent) any number of a variety of diseases or conditions, such as any of the diseases or conditions described above. In embodiments where the medical intervention is a surgery, the surgery may affect any organ or organ system of the body. In some instances, the surgery may be, but is not limited to, a cesarean section, organ replacement, joint replacement, hysterectomy, heart surgery, bariatric surgery, organ or tumor removal, brain surgery, etc. In embodiments where the medical intervention is a therapy, the therapy may be, but is not limited to, psychotherapy (e.g., psychodynamic therapy or behavioral therapy), physical therapy, or gene therapy.

In some embodiments, the medical intervention may include a lifestyle change such as, e.g., an exercise plan, a career change, or a dietary change or restriction. In some embodiments, the medical intervention may include a temporary or permanent modification to the subject's responsibilities of employment. In some instances, the medical intervention may include a detoxification process or the wearing of personal protective equipment (PPE).

As described above, the IPD may be associated with individuals exposed to or undergoing a medical intervention (such as, e.g., any of the medical interventions described above). In some instances, the IPD regards individuals exposed to or undergoing the same medical intervention. In other instances, the IPD may pertain to individuals exposed to or undergoing a variety of different medical interventions. In these cases, the individuals may be diagnosed and/or afflicted with the same disease or condition and the medical interventions may be applied to the individuals in order to treat or address the disease or condition. For example, the IPD may regard individuals diagnosed with inflammatory bowel disease (IBD) and may include a plurality of individuals receiving a first steroid-sparing immunosuppressant to address the IBD, and a plurality of individuals receiving a second steroid-sparing immunosuppressant to address the IBD, and a plurality of individuals receiving a third steroid-sparing immunosuppressant to address the IBD, etc.

As described above, embodiments of the methods include selecting one or more patient-level features and an outcome variable enabled by the IPD. By patient-level feature is meant a characteristic or attribute that may be specific to an individual such as, e.g., each individual of a clinical trial from which the IPD was sourced. The patient-level feature may include categorical variables and/or continuous variables. The patient-level feature may be enabled by the IPD if a certain number or proportion of the individuals comprising the IPD include data pertaining to the patient-level features is sufficient to generate a fitted placebo or medical intervention regression model that meets a standard or threshold of statistical relevance (e.g., as determined by a statistical test such as a T-test or an ANOVA test) for a specific application, as is described in greater detail below.

In some embodiments, the one or more patient level features are selected based on the number of individuals of the obtained IPD for which the one or more patient level features are included. In some cases, the one or more patient level features are selected based on the disease or condition and/or the medical intervention. In these instances, one or more of the patient level features may be selected using domain specific knowledge. For example, one or more of the patient level features may be selected by a medical professional or medical researcher. The one or more of the patient level features may be associated with demographic information, chronic disease burden, a sign or symptom of the disease or condition, and/or a previously known modifier of response to a given medical intervention.

As described above, embodiments of the methods include processing the obtained IPD. In some embodiments, the processing includes filtering and/or harmonizing the obtained IPD (e.g., a subset of obtained IPD). In some cases, obtained IPD is filtered to include only data generated from clinical trials that are completed, phase 2-4, randomized, double blind, interventional, and/or approved by the U.S. Food and Drug Administration (FDA). In some instances, obtained IPD is filtered to include only data generated from clinical trials including a minimum threshold of continuous observation time of one or more subjects on a placebo relative to a baseline. In these instances, the minimum threshold of time may depend on the disease or condition and/or the medical intervention of the clinical trials associated with the obtained IPD. For example, the minimum threshold of time may be at least a week, at least five weeks, at least three months, at least a year, etc. In some cases, the obtained IPD is filtered to exclude participants for which one or more of the patient level features are not included. In these cases, the patient level features that are not included may be categorical variables.

As discussed above, the processing may include filtering and/or harmonizing the obtained IPD (e.g., a subset of obtained IPD). In some cases, the harmonizing includes imputing one or more of the patient level features and/or the outcome variable for a participant of the obtained IPD missing the one or more patient level features and/or the outcome variable. In some embodiments, the imputing may include median imputation such as, e.g., when the patient level feature and/or the outcome variable is a continuous variable. In embodiments where the outcome variable is a continuous variable, the imputing may include a last observation carried forward (LOCF) imputation. In other instances, the imputing may include replacing the not missing patient level feature using domain specific knowledge.

In some embodiments, the filtering includes performing a quality control evaluation. In some instances, the quality control evaluation may generate a quality metric for each clinical trial. In these cases, the obtained IPD may be filtered to exclude participants associated with clinical trials having a generated quality metric below a predetermined threshold value. The quality control evaluation may include any method or technique of evaluating the quality or reproducibility of a clinical trial known in the art, and combinations thereof.

In some embodiments, all obtained IPD is processed, e.g., as described above. In other embodiments, one or more of the processing methods may be applied only to one or more specific subsets of the IPD. In these instances, the subsets of obtained IPD may include, but are not limited to, e.g., a subset including only IPD associated with individuals that have received a placebo, only IPD associated with individuals that have received a specific medical intervention, etc.

As described above, embodiments of the methods include fitting a regression model to one or processed subsets of IPD (or information/data obtained from processed IPD subsets such as, e.g., medical intervention attributable components of outcome variables, as described below) wherein the one or more patient level features of each individual and the clinical trial associated with each individual are modeled as covariates. The regression models of the disclosure may be mixed effects regression models. Mixed effects regression models, in accordance with embodiments of the methods, may vary and may include, but are not limited to, any of the models discussed below, or any model known in the art capable of performing mixed effects regressions on the variables or data to which the models of the methods are fitted, as discussed herein (e.g., patient level features, outcome variables reflecting the state of a disease or condition within a subject, medical intervention attributable components of said outcome variables, etc.).

In some embodiments, a mixed effects regression model of the methods may include a statistical regression model. In some cases, the statistical regression model may include a linear mixed effects model, a generalized linear mixed effects model, and/or a nonlinear mixed effects model. In some instances, the statistical regression model is a linear mixed effects model. In some embodiments, the statistical regression model (e.g., the linear mixed effects statistical model) may be fit using the R computer programming language (e.g., the 1 me4 package). In these instances, the statistical model may be fit using restricted maximum likelihood (REML), while in other instances other approaches may be used. In some embodiments, a mixed effects regression model of the methods may include a machine learning (ML) model. In some cases, the ML model may include, or be configured to employ, a K-nearest neighbors (KNN), logistic regression, linear discriminant analysis (LDA), artificial neural network (NN), and/or XGBoost Decision Trees (XGBoost) algorithm. In some embodiments, the ML model is a supervised ML model. In these instances, the supervised ML model may include a tree-based model, a Bayesian model, a neural network (NN), and/or Gaussian process model.

In some embodiments of the methods, a mixed effects regression model is fit to a processed subset of IPD including only data associated with individuals that have received a placebo (i.e., a placebo regression model is fit). In these instances, the one or more patient level features of each individual of the processed subset and the clinical trial associated with each individual may be modeled as covariates. In these cases, the plurality of clinical trials associated with the subset of IPD may lack a shared control group. In some cases, the one or more patient level features of each individual are modeled as fixed effects. In some cases, the clinical trial associated with each individual is modeled as a random effect. In some embodiments, the period in time when each clinical trial was performed may be modeled as a covariate. For example, the year in which each clinical trial was performed may be modeled as a fixed effect.

In some embodiments of the methods, a mixed effects regression model is fit for a processed subset of IPD including only data associated with individuals that have received a medical intervention. In these instances, the regression model may be fit to a medical intervention attributable component of the outcome variable for individuals of the processed subset and the processed subset of IPD associated with the medical intervention. In these instances, the medical intervention regression model may be fit using the one or more patient level features of each individual of the processed subset (e.g., modeled as covariates as discussed above). The intervention attributable component of the outcome variable may be determined, e.g., using a placebo regression model as discussed above and the processed subset of IPD associated with the medical intervention.

In some embodiments, the IPD is obtained from a plurality of clinical trials regarding the treatment of a specific disease or condition with one or more different predetermined medical interventions. In these cases, a medical intervention regression model may be fit for each predetermined medical interventions, e.g., as described above.

In some cases, fitted medical intervention regression models may be used to identify relationships between the medical intervention attributable component of the fitted model and the one or more patient level features of the subset of processed IPD used to fit the model. In some cases, embodiments of the methods further include simulating the effect of a medical intervention on an individual of the obtained IPD, wherein the simulated medical intervention is selected from the medical interventions of fitted medical intervention regression models (e.g., as described above). In these instances, the simulation may include: determining a relationship between the value or class of each patient level feature and the medical intervention attributable component of the outcome variable for the selected medical intervention using the medical intervention regression model fitted for the selected medical intervention; and simulating the effect of the selected medical intervention on the individual using the one or more patient level features of the individual and the relationship determined for each patient level feature. In some cases, the effect of a medical intervention may be simulated for a subject or individual having the disease or condition that is not associated with the IPD (e.g., has not participated in a clinical trial). In these instances, the effect of a medical intervention may be simulated for multiple different medical interventions. In these cases, the simulated effects (i.e., for each of the different medical interventions) may be used to rank the medical interventions by safety and/or efficacy for the subject or individual. The ranking of medical interventions may then be used to generate a personalized treatment recommendation including, e.g., the use of one or more of the medical interventions (i.e., associated with the simulations) by the subject.

In some embodiments, fitted medical intervention regression models may be used to generate a personalized treatment recommendation for a subject having a disease or condition and one or more patient level features (i.e., as described above). In these cases, the method may comprise: obtaining individual participant data (IPD) from a plurality of clinical trials regarding the treatment of the disease or condition with multiple different medical interventions, wherein the clinical trials lack a shared control group; processing a subset of the obtained IPD including only data associated with individuals that have received a placebo and a subset of the obtained IPD for each of the multiple different medical interventions including only data associated with individuals that have received a specific medical intervention of the multiple different medical interventions, wherein the processed IPD includes the one or more patient level features and an outcome variable for each individual; and fitting a placebo regression model to the processed placebo subset of IPD in order to determine an effect of each patient level feature on the outcome variable; determining, for each medical intervention subset, a component of the outcome variable attributable to the specific medical intervention of the respective intervention subset for each individual of the subset using the one or more patient level features of each individual and the fitted placebo regression model; fitting, for each medical intervention subset, a medical intervention regression model to the medical intervention attributable component and the one or more patient level features of each individual of the respective intervention subset, wherein the one or more patient level features of each individual of the intervention subset are modeled as covariates; and generating the personalized treatment recommendation for the subject using each medical intervention regression model and the one or more patient level features of the subject.

In some embodiments, generating the personalized treatment recommendation for the subject may comprise simulating an effect of each medical intervention on the outcome variable of each individual of the obtained IPD subsets using the one or more patient level features of each individual and each medical intervention regression model. In these instances, a plurality of simulations may be performed for each individual via bootstrapping such as, e.g., 10,000 simulations, or 15,000 simulations, etc. depending on a desired statistical power to be achieved. In some embodiments, the effects of each medical intervention on the outcome variable of each individual are used to identify subgroups of individuals having a greater response to a specific medical intervention of the multiple different medical interventions. In some embodiments, the subgroups comprise a specific range and/or set of patient level features. In some embodiments, the personalized treatment recommendation may be generated by determining if the subject belongs to one or more of the identified subgroups.

### Systems

Aspects of the present disclosure further include systems, such as computer-controlled systems, for practicing embodiments of the above methods. Aspects of the systems include: a processor including memory operably coupled to the processor, wherein the memory includes instructions stored thereon, which, when executed by the processor, cause the processor to: obtain individual participant data from a plurality of clinical trials regarding the treatment of a disease or condition with a medical intervention, wherein the clinical trials lack a shared control group; select one or more patient level features and an outcome variable enabled by the individual participant data; filter and harmonize a subset of the original patient data including only data associated with individuals that have received a placebo; and fit a regression model to the filtered and harmonized subset of data using the one or more patient level features of each individual. In some embodiments, the memory further includes instructions stored thereon, which, when executed by the processor, cause the processor to: filter and harmonize a second subset of the individual patient data including only data associated with individuals that have received a first medical intervention; determine a component of the outcome variable attributable to the first medical intervention for each individual of the second subset using the one or more patient level features of each individual and the fitted placebo regression model; and fit a medical intervention regression model to the first medical intervention attributable component and the one or more patient level features of each individual of the second subset.

In some instances the systems further include one or more computers for complete automation or partial automation of the methods described herein. In some embodiments, systems include a computer having a computer readable storage medium with a computer program stored thereon.

In embodiments, the system includes an input module, a processing module and an output module. The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, Python, other high-level or low-level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques. The processor may be any suitable analog or digital system. In some embodiments, the processor includes analog electronics which provide feedback control, such as for example negative feedback control.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described including a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid-state storage devices (including magnetic or optical disks or tape or RAM, or any other suitable device, either fixed or portable). The processor may include a general-purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, systems according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, an RS-232 port, or any other suitable electrical connection port to allow data communication between the subject systems and other external devices such as a computer terminal (for example, at a physician's office or in hospital environment) that is configured for similar complementary data communication.

In one embodiment, the communication interface is configured for infrared communication, Bluetooth® communication, or any other suitable wireless communication protocol to enable the subject systems to communicate with other devices such as computer terminals and/or networks, communication enabled mobile telephones, personal digital assistants, or any other communication devices which the user may use in conjunction.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or WiFi connection to the internet at a WiFi hotspot.

In one embodiment, the subject systems are configured to wirelessly communicate with a server device via the communication interface, e.g., using a common standard such as 802.11 or Bluetooth® RF protocol, or an IrDA infrared protocol. The server device may be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen.

In some embodiments, the communication interface is configured to automatically or semi-automatically communicate data stored in the subject systems, e.g., in an optional data storage unit, with a network or server device using one or more of the communication protocols and/or mechanisms described above.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a workstation, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows, iOS, Oracle Solaris, Linux, IBM i, Unix, and others.

Aspects of the present disclosure further include non-transitory computer readable storage mediums having instructions for practicing the subject methods. Computer readable storage mediums may be employed on one or more computers for complete automation or partial automation of a system for practicing methods described herein. In certain embodiments, instructions in accordance with the method described herein can be coded onto a computer-readable medium in the form of “programming”, where the term “computer readable medium” as used herein refers to any non-transitory storage medium that participates in providing instructions and data to a computer for execution and processing. Non-transitory computer-readable media include all computer-readable media except for a transitory, propagating signal. Examples of suitable non-transitory storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Python, Java, Java Script, C, C#, C++, Go, R, Swift, PHP, as well as many others.

The non-transitory computer readable storage medium may be employed on one or more computer systems having a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as those mentioned above, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

### Utility

The methods and systems of the invention, e.g., as described above, find use in a variety of applications where it is desirable to accurately and cost effectively compare treatment efficacy and safety for different medical interventions administered for the treatment of a disease or condition. In some embodiments, the methods and systems described herein find use when it is desirable to improve the design and statistical power of clinical trials across diseases such as, e.g., improving the robustness of potential external control arm studies. In some embodiments, methods and systems described herein find use when it is desirable to characterize or validate a medical intervention recommendation and/or identify previously unrecognized dangers and risks associated with a medical intervention.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-102 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.
1. A method of modeling an effect of a placebo on a subject having a disease or condition, the method comprising:
   obtaining individual participant data (IPD) from a plurality of clinical trials regarding the treatment of the disease or condition with a medical intervention;
   processing a subset of the obtained IPD including only data associated with individuals that have received a placebo, wherein the processed IPD includes one or more patient level features and an outcome variable for each individual; and
   fitting a mixed effects regression model to the processed subset of IPD, wherein the one or more patient level features of each individual and the clinical trial associated with each individual are modeled as covariates.
2. The method according to Aspect 1, wherein the plurality of clinical trials lack a shared control group.
3. The method according to Aspects 1 or 2, wherein the one or more patient level features of each individual are modeled as fixed effects.
4. The method according to any of the preceding aspects, wherein the clinical trial associated with each individual is modeled as a random effect.
5. The method according to any of the preceding aspects, wherein the period in time when each clinical trial was performed is modeled as a covariate.
6. The method according to Aspect 5, wherein the year in which each clinical trial was performed is modeled as a fixed effect.
7. The method according to any of the preceding aspects, wherein the subset of the IPD includes all individuals that have received a placebo.
8. The method according to any of Aspects 1 to 6, wherein the subset of the IPD includes a random sampling of individuals that have received a placebo.
9. The method according to Aspect 8, wherein the random sampling is done with replacement.
10. The method according to any of the preceding aspects, wherein the subject has the disease or condition.
11. The method according to Aspect 10, wherein the disease or condition is a chronic disease or condition.
12. The method according to any of the preceding aspects, wherein the one or more patient level features are selected based on the number of individuals of the obtained IPD for which the one or more patient level features are included.
13. The method according to any of the preceding aspects, wherein the one or more patient level features are selected based on the disease or condition and/or the medical intervention.
14. The method according to Aspect 13, wherein one or more of the patient level features is selected by a medical professional or medical researcher.
15. The method according to any of the preceding aspects, wherein one or more of the patient level features is associated demographic information, chronic disease burden, a sign or symptom of the disease or condition, and/or a previously known modifier of response to the medical intervention.
16. The method according to any of the preceding aspects, wherein the processing comprises filtering and/or harmonizing the subset of obtained IPD.
17. The method according to Aspect 16, wherein the subset of obtained IPD is filtered to include only data generated from clinical trials that are completed, phase 2-4, randomized, double blind, interventional, and/or approved by the U.S. Food and Drug Administration (FDA).
18. The method according to Aspects 16 or 17, wherein the subset of obtained IPD is filtered to include only data generated from clinical trials including a minimum threshold of continuous observation time of one or more subjects on a placebo relative to a baseline.
19. The method according to Aspect 18, wherein the minimum threshold of time is five weeks.
20. The method according to any of Aspects 16 to 19, wherein the subset of obtained IPD is filtered to exclude participants for which one or more of the patient level features are not included.
21. The method according to Aspect 20, wherein the patient level feature not included is a categorical variable.
22. The method according to any of Aspects 16 to 21, wherein the harmonizing comprises imputing one or more of the patient level features and/or the outcome variable for a participant of the obtained IPD missing the one or more patient level features and/or the outcome variable.
23. The method according to Aspect 22, wherein the imputing comprises median imputation of when the patient level feature and/or the outcome variable is a continuous variable.
24. The method according to Aspect 22, wherein the imputing comprises a last observation carried forward (LOCF) imputation.
25. The method according to Aspect 22, wherein the imputing comprises replacing the missing patient level feature using domain specific knowledge.
26. The method according to any of Aspects 16 to 25, wherein the filtering comprises performing a quality control evaluation.
27. The method according to Aspect 26, wherein the quality control evaluation comprises generating a quality metric for each clinical trial.
28. The method according to Aspect 27, wherein the obtained IPD is filtered to exclude participants associated with clinical trials having a generated quality metric below a predetermined threshold value.
29. The method according to any of Aspects 27 to 28, wherein the quality control evaluation comprises reproducing one or more published statistics for each clinical trial.
30. The method according to any of the preceding aspects, wherein the mixed effects regression model comprises a statistical regression model and/or a machine learning (ML) model.
31. The method according to Aspect 30, wherein the mixed effects regression model comprises a statistical regression model.
32. The method according to Aspect 31, wherein the mixed effects regression model comprises a linear mixed effects regression model.
33. The method according to Aspect 31 or 32, wherein the statistical model is fit using the R computer programming language.
34. The method according to Aspect 30, wherein the mixed effects regression model comprises a supervised ML model.
35. The method according to Aspect 34, wherein the supervised ML model comprises a tree-based model, a Bayesian model, a neural network (NN), and/or a Gaussian process model.
36. The method according to any of the preceding aspects, wherein the disease or condition is a polygenetic disease.
37. The method according to Aspect 36, wherein the disease or condition is Crohn's disease.
38. The method according to Aspect 37, wherein the medical intervention comprises anti-TNF, anti-integrins, or anti-IL-12/23.
39. The method according to any one of the preceding aspects, wherein the disease or condition is a hereditary disease.
40. The method according to Aspect 39, wherein the disease or condition is acute hepatic porphyria, primary hyperoxaluria, or hereditary transthyretin amyloidosis.
41. The method according to any one of the preceding aspects, wherein the medical intervention comprises a pharmaceutical composition, a medical device, a surgery, and/or a therapy.
42. The method according to Aspect 40, wherein the medical intervention is a pharmaceutical composition.
43. The method according to any one of the preceding aspects, wherein the IPD is obtained from a database.
44. The method according to Aspect 43, wherein the database is the Vivli data-sharing platform or the Yale University Open Data Access (YODA) project.
45. The method according to any one of the preceding aspects, further comprising designing a clinical trial using the fitted regression model.
46. The method according to any one of the preceding aspects, further comprising identifying a relationship between the outcome variable and one or more of the patient specific features using the fitted regression model.
47. The method according to Aspect 46, wherein one or more of the patient specific features is identified as a positive predictor of the placebo effect.
48. The method according to Aspect 46 or 47, wherein one or more of the patient specific features is identified as a negative predictor of the placebo effect.
49. The method according to any one of the preceding aspects, further comprising processing a second subset of the IPD including only data associated with individuals that have received a first medical intervention.
50. The method according to Aspect 49, further comprising determining a component of the outcome variable attributable to the first medical intervention for each individual of the second subset using the one or more patient level features of each individual and the fitted regression model.
51. The method according to Aspect 50, further comprising fitting a medical intervention regression model to the first medical intervention attributable component and the one or more patient level features of each individual of the second subset.
52. The method according to Aspect 51, wherein the fitted medical intervention regression model is used to identify relationships between the first medical intervention attributable component and the one or more patient level features.
53. The method according to Aspect 52, wherein one or more of the patient specific features is identified as a positive predictor of patient response to the first medical intervention.
54. The method according to Aspect 52 or 53, wherein one or more of the patient specific features is identified as a negative predictor of patient response to the first medical intervention.
55. A method of modeling an effect of a medical intervention on a subject having a disease or condition, the method comprising:
   obtaining individual participant data (IPD) from a plurality of clinical trials regarding the treatment of the disease or condition with one or more medical interventions, wherein the clinical trials lack a shared control group;
   processing a first subset of the obtained IPD including only data associated with individuals that have received a placebo and a second subset of the obtained IPD including only data associated with individuals that have received a first medical intervention, wherein the processed IPD includes one or more patient level features and an outcome variable for each individual; and
   fitting a placebo regression model to the processed first subset of IPD in order to determine an effect of each patient level feature on the outcome variable;
   determining a component of the outcome variable attributable to the first medical intervention for each individual of the processed second subset using the one or more patient level features of each individual and the fitted placebo regression model;
   fitting a first medical intervention regression model to the first medical intervention attributable component and the one or more patient level features of each individual of the second subset, wherein the one or more patient level features of each individual of the second subset are modeled as covariates.
56. The method according to Aspect 55, wherein the IPD is obtained from a plurality of clinical trials regarding the treatment of the disease or condition with one or more predetermined medical interventions different from the first medical intervention.
57. The method according to any Aspect 56, wherein the method further comprises:
   processing a subset of the obtained IPD consisting of data associated with individuals that have received a predetermined medical intervention;
   determining a component of the outcome variable attributable to the predetermined medical intervention for each individual of the processed predetermined medical intervention subset using the one or more patient level features of each individual and a fitted placebo regression model; and
   fitting a medical intervention regression model to the predetermined medical intervention attributable component and the one or more patient level features of each individual of the processed predetermined medical intervention subset of IPD, wherein the one or more patient level features of each individual of the processed predetermined medical intervention subset are modeled as covariates;
for each of the predetermined medical interventions.
58. The method according to Aspect 57, wherein for one of the processed predetermined medical intervention subsets the fitted placebo regression model used to determine the component of the outcome variable attributable to the predetermined medical intervention for each individual of the subset is fitted using the processed first subset of IPD.
59. The method according to Aspect 57, wherein for one of the processed predetermined medical intervention subsets the fitted placebo regression model used to determine the component of the outcome variable attributable to the predetermined medical intervention for each individual of the subset is fitted using a third subset of the IPD including only data associated with individuals that have received a placebo.
60. The method according to any one of Aspects 57 to 59, wherein the processed first subset of the IPD includes all individuals that have received a placebo, the processed second subset of the IPD includes all individuals that have received the first medical intervention, and/or one of the processed predetermined medical intervention subsets of the IPD includes all individuals that have received the predetermined medical intervention of the subset.
61. The method according to any one of Aspects 57 to 59, wherein the processed first subset of the IPD includes a random sampling of individuals that have received a placebo, the processed second subset of the IPD includes a random sampling of individuals that have received the first medical intervention, and/or one of the processed predetermined medical intervention subsets of the IPD includes a random sampling of individuals that have received the predetermined medical intervention of the subset.
62. The method according to Aspect 61, wherein the random sampling is done with replacement.
63. The method according to any one of Aspects 55 to 62, wherein each regression model is a mixed effects regression model.
64. The method according to Aspect 63, wherein the one or more patient level features of each individual are modeled as fixed effects.
65. The method according to Aspect 63 or 64, wherein the clinical trial associated with each individual is modeled as a random effect covariate.
66. The method according to any one of Aspects 63 to 65, wherein the period in time when each clinical trial was performed is modeled as a covariate.
67. The method according to Aspect 66, wherein the year in which each clinical trial was performed is modeled as a fixed effect.
68. The method according to any one of Aspects 63 to 67, wherein the subject has the disease or condition.
69. The method according to Aspect 68, wherein the disease or condition is a chronic disease or condition.
70. The method according to any one of Aspects 63 to 69, wherein the one or more patient level features are selected based on the number of individuals of the obtained IPD for which the one or more patient level features are included.
71. The method according to Aspect 70, wherein the one or more patient level features are selected based on the disease or condition and/or the medical intervention.
72. The method according to Aspect 71, wherein one or more of the patient level features is selected by a medical professional or medical researcher.
73. The method according to any one of Aspects 63 to 72, wherein one or more of the patient level features is associated demographic information, chronic disease burden, a sign or symptom of the disease or condition, and/or a previously known modifier of response to each medical intervention.
74. The method according to any one of Aspects 63 to 73, wherein the processing comprises filtering and/or harmonizing each subset of obtained IPD.
75. The method according to Aspect 74, wherein each subset of obtained IPD is filtered to include only data generated from clinical trials that are completed, phase 2-4, randomized, double blind, interventional, and/or approved by the U.S. Food and Drug Administration (FDA).
76. The method according to Aspect 74 or 75, wherein each subset of obtained IPD is filtered to include only data generated from clinical trials including a minimum threshold of continuous observation time of one or more subjects on a placebo and/or a medical intervention relative to a baseline.
77. The method according to Aspect 76, wherein the minimum threshold of time is five weeks.
78. The method according to any one of Aspects 74 to 77, wherein each subset of obtained IPD is filtered to exclude participants for which one or more of the patient level features are not included.
79. The method according to Aspect 78, wherein the patient level feature not included is a categorical variable.
80. The method according to any one of Aspects 74 to 79, wherein the harmonizing comprises imputing one or more of the patient level features and/or the outcome variable for a participant of the obtained IPD missing the one or more patient level features and/or the outcome variable.
81. The method according to Aspect 80, wherein the imputing comprises median imputation when the missing patient level feature and/or the outcome variable is a continuous variable.
82. The method according to Aspect 80, wherein the imputing comprises a last observation carried forward (LOCF) imputation.
83. The method according to Aspect 80, wherein the imputing comprises replacing the missing patient level feature using domain specific knowledge.
84. The method according to any one of Aspects 74 to 83, wherein the filtering comprises performing a quality control evaluation.
85. The method according to Aspect 84, wherein the quality control evaluation comprises generating a quality metric for each clinical trial.
86. The method according to Aspect 85, wherein the obtained IPD is filtered to exclude participants associated with clinical trials having a generated quality metric below a predetermined threshold value.
87. The method according to any one of Aspects 84 to 86, wherein the quality control evaluation comprises reproducing one or more published statistics for each clinical trial.
88. The method according to any one of Aspects 63 to 87, wherein each mixed effects regression model comprises a statistical regression model and/or a machine learning (ML) model.
89. The method according to Aspect 88, wherein one or more of the mixed effects regression models comprise a statistical regression model.
90. The method according to Aspect 89, wherein the one or more statistical regression models comprise linear mixed effects regression models.
91. The method according to Aspect 89 or 90, wherein each statistical model is fit using the R computer programming language.
92. The method according to Aspect 88, wherein one or more of the mixed effects regression models comprise a supervised ML model.
93. The method according to Aspect 92, wherein the one or more supervised ML models comprise tree-based models, Bayesian models, a neural networks (NNs), and/or Gaussian process models.
94. The method according to any one of Aspects 55 to 93, wherein the disease or condition is a polygenetic disease.
95. The method according to Aspect 94, wherein the disease or condition is Crohn's disease.
96. The method according to Aspect 95, wherein each medical intervention comprises anti-TNF, anti-integrins, or anti-IL-12/23.
97. The method according to any one of Aspects 55 to 93, wherein the disease or condition is a hereditary disease.
98. The method according to Aspect 97, wherein the disease or condition is acute hepatic porphyria, primary hyperoxaluria, or hereditary transthyretin amyloidosis.
99. The method according to any one of Aspects 55 to 98, wherein each medical intervention comprises a pharmaceutical composition, a medical device, a surgery, and/or a therapy.
100. The method according to Aspect 99, wherein each medical intervention is a pharmaceutical composition.
101. The method according to any one of Aspects 55 to 100, wherein the IPD is obtained from a database.
102. The method according to Aspect 101, wherein the database is the Vivli data-sharing platform or the Yale University Open Data Access (YODA) project.
103. The method according to any one of Aspects 55 to 102, further comprising designing a clinical trial using one or more of the fitted regression models.
104. The method according to any one of Aspects 55 to 103, wherein the first fitted medical intervention regression model is used to identify relationships between the first medical intervention attributable component and the one or more patient level features.
105. The method according to any one of Aspects 57 to 104, wherein one or more of the predetermined medical intervention regression models are used to identify relationships between the predetermined medical intervention attributable component of the model and the one or more patient level features.
106. The method according to any one of Aspects 57 to 105, wherein IPD is obtained from a plurality of clinical trials regarding the treatment of the disease or condition with two or more different predetermined medical interventions.
107. The method according to any one of Aspects 57 to 106, wherein IPD is obtained from a plurality of clinical trials regarding the treatment of the disease or condition with three or more different predetermined medical interventions.
108. The method according to any one of Aspects 57 to 107, wherein the predetermined medical interventions are determined by a medical professional for a subject having the disease or condition.
109. The method according to any one of Aspects 57 to 108, further comprising simulating the effect of a medical intervention on an individual of the obtained IPD, wherein the simulated medical intervention is selected from the medical interventions of the fitted medical intervention regression models.
110. The method according to Aspect 109, wherein the simulation comprises:
   determining a relationship between the value or class of each patient level feature and the medical intervention attributable component of the outcome variable for the selected medical intervention using the medical intervention regression model fitted for the selected medical intervention;
   simulating the effect of the selected medical intervention on the individual using the one or more patient level features of the individual and the relationship determined for each patient level feature.
111. A method of generating a personalized treatment recommendation for a subject having a disease or condition and one or more patient level features, the method comprising:
   obtaining individual participant data (IPD) from a plurality of clinical trials regarding the treatment of the disease or condition with multiple different medical interventions, wherein the clinical trials lack a shared control group;
   processing a subset of the obtained IPD including only data associated with individuals that have received a placebo and a subset of the obtained IPD for each of the multiple different medical interventions including only data associated with individuals that have received a specific medical intervention of the multiple different medical interventions, wherein the processed IPD includes the one or more patient level features and an outcome variable for each individual; and
   fitting a placebo regression model to the processed placebo subset of IPD in order to determine an effect of each patient level feature on the outcome variable;
   determining, for each medical intervention subset, a component of the outcome variable attributable to the specific medical intervention of the respective intervention subset for each individual of the subset using the one or more patient level features of each individual and the fitted placebo regression model;
   fitting, for each medical intervention subset, a medical intervention regression model to the medical intervention attributable component and the one or more patient level features of each individual of the respective intervention subset, wherein the one or more patient level features of each individual of the intervention subset are modeled as covariates; and
   generating the personalized treatment recommendation for the subject using each medical intervention regression model and the one or more patient level features of the subject.
112. The method according to Aspect 111, wherein each regression model is a mixed effect regression model.
113. The method according to Aspect 112, wherein the one or more patient level features of each individual are modeled as fixed effects.
114. The method according to Aspect 112 or 113, wherein the clinical trial associated with each individual is modeled as a random effect covariate.
115. The method according to any one of Aspects 112 to 114, wherein the period in time when each clinical trial was performed is modeled as a covariate.
116. The method according to any one of Aspects 112 to 115, wherein generating the personalized treatment recommendation for the subject comprises simulating an effect of each medical intervention on the outcome variable of each individual of the obtained IPD subsets using the one or more patient level features of each individual and each medical intervention regression model.
117. The method according to Aspect 116, wherein a plurality of simulations are performed for each individual via bootstrapping.
118. The method according to Aspect 116 or 117, wherein the effects of each medical intervention on the outcome variable of each individual are used to identify subgroups of individuals having a greater response to a specific medical intervention of the multiple different medical interventions.
119. The method according to Aspect 118, wherein the subgroups comprise a specific range and/or set of patient level features.
120. The method according to Aspect 118 or 119, wherein the personalized treatment recommendation is generated by determining if the subject belongs to one or more of the identified subgroups.
121. The method according to any one of Aspects 112 to 120, wherein each mixed effects regression model comprises a statistical regression model and/or a machine learning (ML) model.
122. The method according to Aspect 121, wherein one or more of the mixed effects regression models comprise a statistical regression model.
123. The method according to Aspect 122, wherein the one or more statistical regression models comprise linear mixed effects regression models.
124. The method according to Aspect 122 or 123, wherein each statistical model is fit using the R computer programming language.
125. The method according to Aspect 121, wherein one or more of the mixed effects regression models comprise a supervised ML model.
126. The method according to Aspect 125, wherein the one or more supervised ML models comprise tree-based models, Bayesian models, a neural networks (NNs), and/or Gaussian process models.

### EXPERIMENTAL

As demonstrated in the above disclosure, the present invention has a wide variety of applications. The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Efforts have been made to ensure accuracy with respect to numbers used (e.g. amounts, percentages, etc.) but some experimental errors and deviations should be accounted for.

### Example 1: Estimating Causal Effects from Heterogeneous Clinical Trials without a Common Control Group Using Sequential Regression and Simulation

### Introduction

The individual participant data (IPD) meta-analysis is the gold-standard for clinical research [1,2]. Access to the raw data from trials affords investigators the opportunity to verify published results, ask new questions of these data, and uncover findings with the potential to impact patient care. Performing an IPD meta-analyses usually requires multiple trials with negligible heterogeneity across many dimensions: cohort definition, randomization, blinding, parallel study arms, interventions, and outcomes [1,2]. Although this requirement ensures unbiased estimation, it substantially limits the number of meta-analyses that can be performed due to the rarity of replicate trials.

The method of mixed-effects regression may be used to address study heterogeneity when meta-analyzed trials include a shared control group (i.e. placebo). However, there is a paucity of methods for common situations where there is no shared control group across potential studies. The few methods that have been developed include naïve pooling [3], as well as the Bayesian method of power priors [4-7]. However, these methods fail to address the problem of cohort heterogeneity [8, 9]. Another major limitation is the lack of external validation against prospective studies. The result of this methodological gap is the common practice of excluding uncontrolled studies from potential meta-analyses and, ultimately, being statistically powered to answer fewer research using already-collected data.

Here a new method for meta-analyzing clinical trials data in the absence of a common control group is reported. A method of sequential regression and simulation is illustrated in the context of a comparative efficacy analysis in Crohn's disease, an immune disorder of the gastrointestinal tract. The data from six placebo-controlled trials (N=3153) is used to develop a model of the placebo effect, then applied to three placebo-less trials (N=239) to normalize and separately model the drug-attributable response. Finally, the method is validated by predicting the results of SEAVUE (NCT03464136), a recent head-to-head trial of ustekinumab versus adalimumab [10].

### Data Access

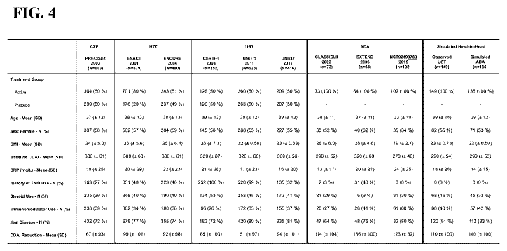

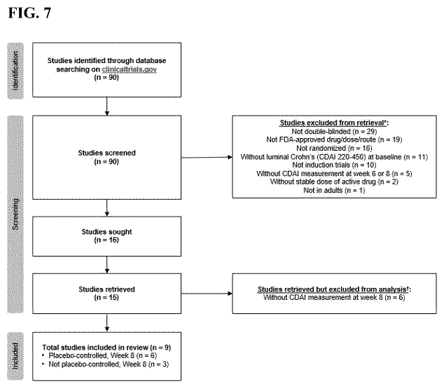

In June 2019 clinicaltrials.gov was queried to identify studies for meta-analysis (FIG. 7, FIG. 4). 90 trials listed as completed, phase 2-4, randomized, double-blinded, interventional trials of FDA-approved treatments for Crohn's disease were found. 16 trials were manually confirmed as meeting these criteria. To ensure comparability of these studies, their major inclusion and exclusion criteria were reviewed and it was confirmed that the Crohn's Disease Activity Index (CDAI) had been captured at week six or eight relative to treatment initiation. These studies were confirmed to have low risk of bias using the Cochrane ROB2 tool. Access to the IPD was obtained for 15 studies (N=5703). They were conducted between 1999 and 2015 and corresponded to all six FDA-approved biologics as of 2019.

### Study Design

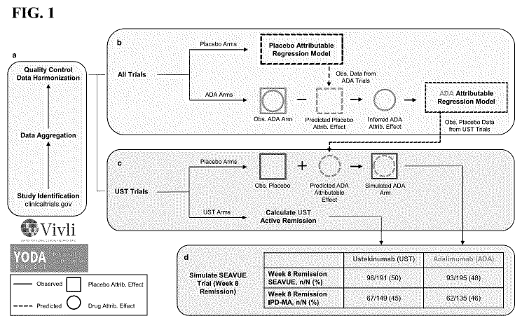

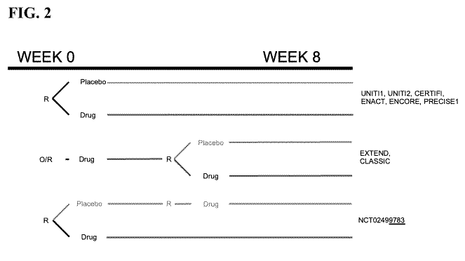

This study was designed emulate a hypothetical head-to-head, parallel-design, efficacy trial randomizing participants to two treatment arms (FIG. 1). Although a typical meta-analytic study design would have involved pooling cohorts from several internally controlled, parallel-arm trials, this was not possible in this case for several reasons. For example, many studies involved open-label induction followed by a randomization event to continue or discontinue the treatment (FIG. 2).

To overcome this heterogeneity, the primary outcome was defined as the absolute reduction in CDAI at week eight, and the provided data was filtered to include only those cohorts that had at least eight weeks of uninterrupted observation time on either placebo or a drug relative to baseline. It was noted that nine of the 15 trials did not include a parallel arm placebo cohort randomized at week 0 and followed for eight weeks. Thus, for this study, they were considered uncontrolled. As a first step towards developing a method for handling this heterogeneity, the initial analyses was restricted to just the placebo-controlled trials (six trials; N=3153; FIG. 4). Subsequent analyses used a second set of placebo-less trials of adalimumab, one of the drugs compared in SEAVUE.

### Quality Control, Harmonization

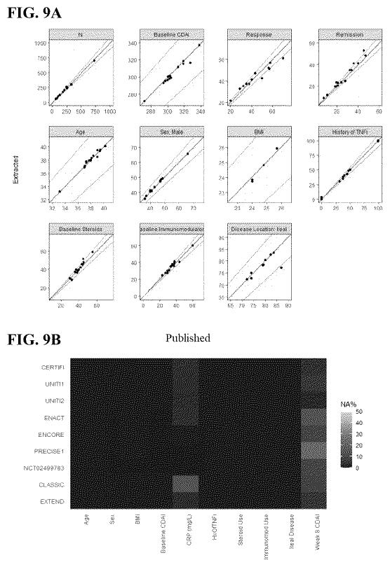

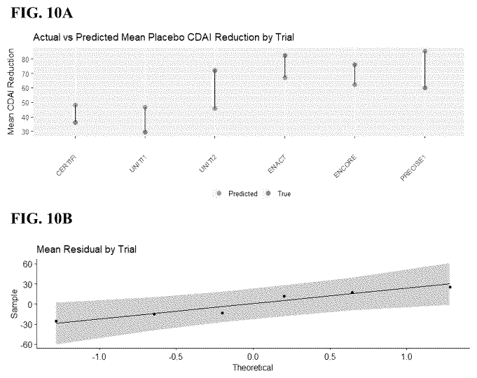

Extensive tests of data quality were performed (Methods). These included reproducing published results from each trial cohort (FIGS. 9 and 10). Domain knowledge was used to select nine variables that were universally available across trials for subsequent modeling: Age, Sex, body mass index (BMI), baseline CDAI, c-reactive protein (CRP), history of tumor necrosis factor-alpha inhibitor (TNFi) use, steroid use, immunomodulator use, and ileal involvement.

3% of the participants had at least one missing covariate at baseline. Continuous variables were addressed by median imputation, and participants with missing categorical variables were dropped (N=86). 11% of participants had a missing outcome at week eight. Last-observation-carried-forward was used to impute these. This is the typical practice for the analysis of these trials in regulatory submissions and was the prespecified approach in the protocols of the included trials.

### Modeling Assumptions

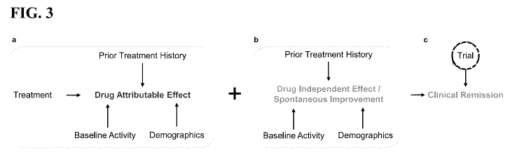

Several assumptions were incorporated when developing and interpreting candidate models. It was assumed that the observed week eight reduction in CDAI reflected a combination of two distinct effects: a drug-independent (i.e., placebo) effect and drug-attributable effect. These effects were separately modeled as a function of the above predictors and study year. The justification for this is briefly summarized below and additionally presented graphically (FIG. 3).

The placebo effect was modeled as a function of the nine covariates as well as predictors of trial-specific heterogeneity. It was assumed that much of the spontaneous improvement seen in placebo-assigned participants was related to regression to the mean, as study participation was limited to patients with currently active Crohn's disease. Conversely, it was assumed that failure to spontaneously improve was likely to reflect chronic and cumulative disease burden with relative stability in symptoms. Thus, variables corresponding to concomitant and prior treatments were treated as proxies of chronic disease burden and included as predictors. Lastly, other influences were considered on overall heterogeneity, including differences in cohorts, data capture, outcome ascertainment, and study personnel. To account for these sources of variation, study year as well as trial identifier were included as additional covariates. In mixed-effect models, trial was included as a random effect. Other covariates were fixed effects.

The drug-attributable effect was separately modeled as a function of these same covariates, reflecting drug-specific (interaction) effects on the outcome. Many of these covariates are well-established as modifiers of treatment response, such as a history of TNFi and immunomodulators use [4]. Others (CRP, baseline CDAI) are proxies of bowel inflammation, the target of these medications. These variables were included to maximize the explained variation in the outcome.

### Development and Assessment of a Placebo Model

A linear mixed effects model was fit utilizing all nine predictors as well as study year as predictors of the placebo effect. To minimize the risks of residual bias due to model misspecification (e.g., non-linearities, unmodeled interactions), the predictive performance of this model was compared against other statistical and machine learning models. This model was further evaluated from the perspective of being used to impute unmeasured placebo effects, and thus normalize different trials to the same background. A leave-one-trial-out analysis was performed, and the trial-averaged residuals were inspected.

### Estimation of the Drug-Attributable Effect

To normalize the responses of drug-assigned cohorts that lacked a within-study, parallel-arm control group, the finalized placebo model was used to simulate and subsequently partition their overall response into drug-independent and drug-dependent (i.e., drug attributable) components (FIG. 1B). This was performed using the data from the adalimumab trials (N=239) because they were all lacking an 8-week continuous placebo group and thus required normalization. From the outcomes of these patients, the conditional mean outcomes associated with the placebo effect were subtracted and the residuals were used as the new outcome variable of a second mixed-effects model to estimate the adalimumab-attributable effect.

### Validation, Sensitivity Analyses

Using the covariates associated with the placebo recipients of the ustekinumab trials, the adalimumab-attributable regression model was used to simulate their counterfactual week 8 outcomes had they received adalimumab instead (FIG. 1C). The subset of these virtual patients were identified who were naïve to TNFi (an additional inclusion criterion from SEAVUE) and the same was done with the ustekinumab recipients. Their week 8 outcomes were compared using the same definition of clinical remission as used in SEAVUE (CDAI<150) and a Fisher's exact test was performed to compare the results with SEAVUE's. The robustness of the result was tested using three sensitivity analyses: 1) removing ENACT and ENCORE from the dataset due to >10% missingness of outcome data, 2) removing participants with missing outcomes, and 3) removing the ustekinumab trials from the placebo model training data, to address potential information leakage. Lastly, the results were compared with what might have been found if the method described herein was not used to normalize cohorts.

### Results

See FIG. 1 for an overview. This method was originally developed in the context of an existing effort to study comparative efficacy in Crohn's disease by reanalyzing the IPD of corresponding clinical trials. As the first step towards this goal, it was sought to address the problem of meta-analyzing data from several potentially heterogeneous trials lacking a common control group.

### Data Access

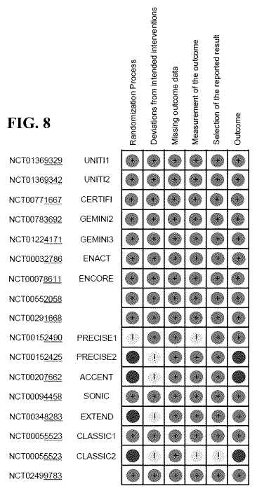

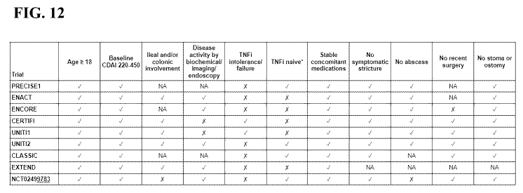

Clinicaltrials.gov was queried and a manual review was performed to confirm 16 trials as meeting these criteria: completed, phase 2-4, randomized, double-blinded, interventional trials of FDA-approved treatments for Crohn's disease as of June 2019 (FIG. 1A, FIG. 7). Included trials had common inclusion/exclusion criteria or had participant-level data available to control for this heterogeneity (FIG. 12). They all measured the same endpoint (CDAI) at week eight and were at low risk of bias (FIG. 8). Access was obtained to the IPD for 15 studies (N=5703), corresponding to trials of all six FDA-approved biologics as of 2019.

### Development and Assessment of a Placebo Model

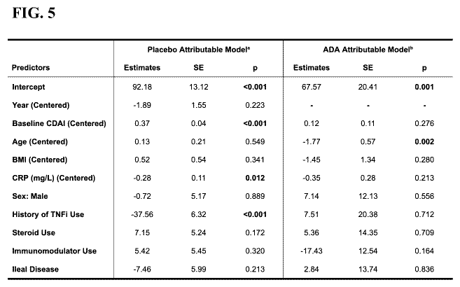

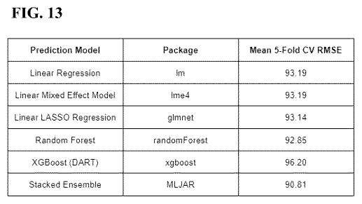

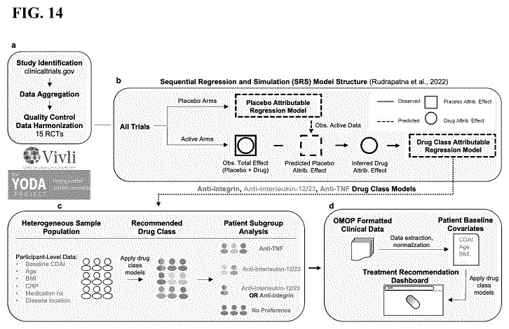

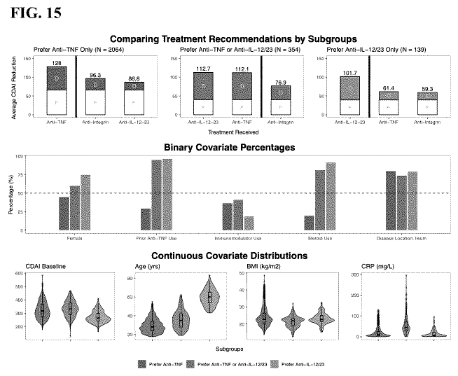

A linear mixed effects model was fit utilizing nine clinical features and study year as predictors of the placebo effect (FIG. 1B, FIG. 5). To minimize the risk of residual bias due to model misspecification, the predictive performance of this model was compared against other machine learning models (FIG. 13). No significant differences were found in the root-mean-squared-error. Thus, the mixed-effects model was selected for downstream analyses.

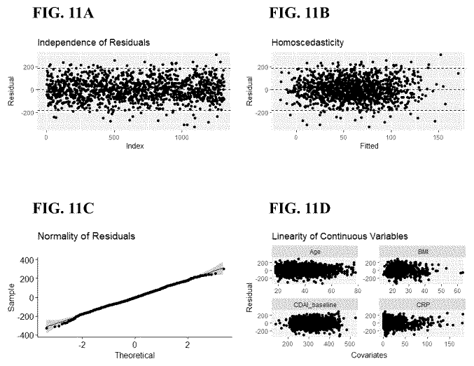

This model was evaluated from the perspective of being used to impute unmeasured placebo effects, and thus normalize different trials to the same background placebo response. A leave-one-trial-out analysis suggested that the model predictions were robust and unbiased (FIGS. 10 and 11). The trial-averaged residuals were consistent with normality (p=0.4; Shapiro-Wilk test).

It was noted that the unmodeled variation in the placebo effect was relatively large and was independent of the choice of model (FIG. 13). These results explain the large placebo effects that have been seen in Crohn's disease randomized trials (regression to the mean) and suggest that more work will be needed to improve the measurement of Crohn's disease activity.

To study the placebo effect and identify potential opportunities to improve trial efficiency, all significant predictors were reviewed. A history of TNFi was associated with a 38-point reduction in the placebo effect. This was interpreted as reflecting a greater cumulative disease burden in patients who failed to improve with TNFis, with disease complications (e.g., minor intestinal strictures) that are unlikely to spontaneously regress over 8 weeks. Similarly, CRP was a negative predictor, suggesting that untreated acute inflammation is unlikely to improve over short time periods. The baseline CDAI was a positive predictor, likely reflecting regression to the mean effects. Age, sex, BMI, concomitant medications, and ileal involvement were not found significant, potentially due to multicollinearity.

### Estimation of the Drug-Attributable Effect

It was sought to normalize the responses of drug-assigned cohorts that lacked a within-study, parallel-arm control group. The strategy was to use the finalized placebo model to partition the overall response into drug-independent and drug-attributable components (FIG. 1B). This approach was applied to the data from three study cohorts assigned to receive adalimumab at the FDA-approved dose for treatment induction (N=239; FIG. 4). This medication was selected because it is one of the two treatments that were compared against each other in SEAVUE, the target of the emulation and validation efforts.

The coefficients of the fitted placebo model were used to predict and remove the placebo-attributable component from the observed outcomes of these participants. The residuals from this process were interpreted as reflecting the adalimumab-attributable effect (FIG. 1B). Across these patients the mean drug-attributable CDAI reduction was 68 points. These residuals were used to fit a second model for the adalimumab-attributable effect (FIG. 5).

As an exploratory analysis the significant predictors of a response to adalimumab were reviewed and these were compared to the corresponding results from the placebo model.

Although the sample size was relatively small, a strong signal for age as a negative predictor was noted: additional decades of life were associated with an 18-point reduction in the response to adalimumab. Interestingly, the direction of this effect was the opposite of that seen in the placebo-only model, suggesting that this coefficient might not have been identified as significant had it not been handled as an interaction term as it was.

### External Validation

To validate the method, an in-silico study was designed to emulate SEAVUE, the only head-to-head study of FDA-approved biologics for Crohn's disease to date [3]. In SEAVUE, biologic-naive patients with active Crohn's disease were randomly selected to receive either adalimumab or ustekinumab as treatment. The primary endpoint was clinical remission at week 52, defined as a CDAI less than 150. Secondary endpoints included clinical remission at the time of all study visits, including week eight.

All participants were identified from the three ustekinumab-related trials who were biologic-naive. 149 subjects were identified who were assigned to ustekinumab and 135 participants assigned to placebo. It was noted that the observed responses of the 135 placebo recipients reflected a combination of individual-specific variability and trial-specific variability (FIG. 3). It was therefore reasoned that to simulate the effect of treatment assignment, it was needed to ‘add back’ the conditional mean effect associated with adalimumab to the outcomes of the placebo recipients (FIG. 1C). Using the model coefficients identified in the adalimumab-attributable regression model (FIG. 5), this extra reduction in the CDAI was computed and added to the observed week eight outcomes of the placebo cohort.

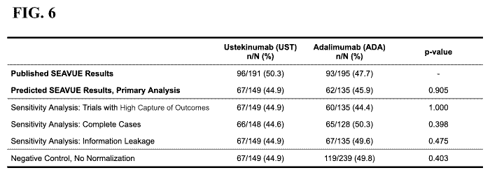

Finally, the proportion of patients who were in clinical remission at week eight was computed, comparing the results of the observed ustekinumab recipients with that of the patients simulated to have received adalimumab and subject to the same background placebo effect (FIG. 1D). It was found that ustekinumab and adalimumab appeared to be equally efficacious, with 45% and 46% of the cohorts in remission. This result closely matched that of SEA VUE (p=0.9), which found 50% and 48% of these corresponding cohorts in remission (FIG. 6). The simulated trial was similar in sample size to SEAVUE, with 149 and 135 patients receiving ustekinumab and adalimumab in the present study, compared to 191 and 195 in SEAVUE.

The robustness of this result was tested using three sensitivity analyses. In the first two trials (PRECISE1, ENACT) associated with the greatest degree of outcome missing data were removed (FIG. 9). In the second, a complete case analysis was performed (deleted patient data associated with missing outcomes) as an alternative to last-observation-carried-forward imputation. In the third all participant data emanating from an ustekinumab trial was removed from the placebo training data, to address a possibility of information leakage. The results remained unchanged over all sensitivity analyses (FIG. 6), supporting the robustness of the primary findings as well as the validity of the overall methodology.

Finally, it was sought to evaluate the value of using the above-described modeling approach compared to another simpler approach using published trial results. One barrier noted to the latter was that the aggregated response of the TNFi-naive subcohorts at week eight was only published in one out of the six trials that was included for this comparison of ustekinumab and adalimumab, making it impossible to emulate SEA VUE using this approach. Separate from this, and to specifically evaluate the value of normalizing disparate cohorts using placebo models, the potential results of the head-to-head assessment was simulated without a normalization step. Under this scenario, the unnormalized adalimumab cohort response was 50% (FIG. 6). While this was not statistically significant compared to the observed ustekinumab response of 45% (p=0.4), it reflects a trend towards a difference. This was interpreted as reflecting a degree of bias that could plausibly result in false positives in other similar studies not analytically controlled using the methods of the present invention.

### Methods

### Quality Control, Harmonization, Missing Data

Extensive quality control evaluations were performed of the included trials and data (FIG. 1A). This included confirming the ability to reproduce published statistics on the trial cohorts at baseline as well as the study primary endpoint (FIG. 9). Most of the study results were able to be exactly reproduced. Where discrepancies occurred, they were generally minor and fell within a 10% error bound. Any potential major discrepancies were reported. It was attempted to completely eliminate all discrepancies, but this was not possible due a variety of factors, including lack of access to the original analytic code or the complete analytic dataset, and inability to contact the original analysts.

An assessment of data availability was completed for all study variables (FIG. 12). Target variables included demographic features, CDAI at baseline and week eight, baseline inflammatory biomarkers, concomitant steroid and immunomodulator use, history of treatment with tumor necrosis factor-alpha inhibitors (TNFis), and other disease-related features. Nine variables were identified that were universally available across all trials and thus could be used for downstream modeling: Age, Sex, BMI, baseline CDAI, c-reactive protein (CRP), history of TNFi use, oral steroid use, immunomodulator use, and ileal involvement.

Only 3% of the participants had at least one missing covariate at baseline. Continuous variables were addressed by median imputation, and participants with missing categorical variables were dropped from the dataset (N=86). 11% of the participants had a missing value for the outcome at week eight. To handle this, last-observation-carried-forward was used to impute these values, typically using measurements from week six and four. This is the typical practice for the analysis of these trials in regulatory submissions and was the prespecified approach in the protocols for all included trials. The variable corresponding to a history of TNFi use was available in all recent trials that occurred after the approval of the very first TNFi medication. Older trials of the first TNFis commonly excluded patients who had a history of exposure to other drugs from this class but did not include this feature as an actual variable in the data set. In these cases, this variable was deterministically imputed corresponding to no prior use.

Other variables of a priori importance could not be included in this study. Ethnicity was not collected in most trials. Race was missing in some trials, but when it was captured, it reflected significant imbalance (88% of participants were white). Other disease specific variables such as disease behavior and duration were also not uniformly captured across studies and thus could not be included in this meta-analysis.

### Statistical Computing

Programming was performed in the R language, using the packages dplyr [18], lme4 [19], ImerTest [20], data.table [21], ggplot2 [22], ggpubr [23], sjstats [24], patchwork [25], and gridExtra [26]. The analytical code was independently reviewed by multiple individuals.

### Model for Estimating the Placebo Effect

A linear mixed effect model was fit to predict the placebo effect on each patient's CDAI reduction at week 8. The model was trained on the placebo arms of the six placebo-controlled trials. They were denoted as trial 1 to trial 6 to simplify the notation. The CDAI reduction of, patient j from trial i in the placebo arm at week 8 is denoted as yᵢⱼ^{placebo} and assumed to be related to the nine predictors D_{ij,1}, . . . , D_{ij,9}, the centered study year Tᵢ, and the trial-specific random effect Sᵢ as in the following equation:

$${y}_{ij}^{placebo} = {β}_{0} + {β}_{1} {D}_{ij , 1} + {β}_{2} {D}_{ij , 2} + … + {β}_{9} {D}_{ij , 9} + {γ}_{i} {T}_{i} + {S}_{i} + {ϵ}_{ij} , \\ i = 1 , … , 6 ; j = 1 , … , {n}_{i} \qquad ( 1 )$$

Where ϵᵢⱼ˜N (0, σ_{ϵ}²), Sᵢ˜N(0, σₛ₁²), and nᵢ is the sample size of each trial, respectively.

### Model for Estimating Adalimumab Drug-Attributable Effect

After fitting the placebo-effect model, the coefficients of model (1) were used to predict the placebo-attributable component of the observed outcomes of the participants from three study cohorts assigned to receive adalimumab at the FDA-approved dose for treatment induction. They were named as trial 7 to trial 9 to simplify the notations. Denoting the observed CDAI reduction at week 8 of patient j from trial i as yᵢⱼ and the predicted placebo-attributable component as yᵢⱼ^{placebo}, it was assumed the difference ϵᵢⱼ=yᵢⱼ−yᵢⱼ^{placebo} reflects the adalimumab drug-attributable effect and is related to the same nine predictors and trial-specific random effect of each adalimumab trial as in the equation below:

$${ϵ}_{ij} = {θ}_{0} + {θ}_{1} {D}_{ij , 1} + {θ}_{2} {D}_{ij , 2} + … + {θ}_{9} {D}_{ij , 9} + {S}_{i} + {ξ}_{ij} \\ i = 7 , 8 , 9 ; j = 1 , … , {n}_{i} \qquad ( 2 )$$

Where ϵᵢⱼ˜N(0, σ_{ξ}²), Si˜N(0, σₛ₂²), and nᵢ is the sample size of each trial, respectively.

### Model for External Validation

To emulate SEAVUE, all placebo-arm participants were identified from the three ustekinumab-related trials who were biologic-naive as the simulated adalimumab cohort. The observed CDAI reduction of the participants at week 8 are denoted as yₖ^{placebo}, where k=1, . . . , 135. The coefficients of model (2) were then used to predict the adalimumab drug-attributable effect of the simulated cohort and denote it as êₖ. The CDAI reduction at week 8 of each simulated adalimumab participant is calculated by ýₖ=yₖ^{placebo}+êₖ. The number of remission Nᵣₑₘ is calculated by the count of BaselineCDAIₖ−ŷₖ≤150. The remission rate is calculated by Nᵣₑₘ/135.

### SUMMARY

A new method was developed for meta-analyzing individual participant data (IPD) from heterogenous randomized trials lacking a shared control group. The methodology was validated by successfully reproducing a major endpoint of SEAVUE, a recent head-to-head trial of biologic therapies in Crohn's disease [3]. The method involved several steps: identifying and isolating parallel arm cohorts from the available trials, harmonization and quality control, separately modeling the placebo effect from drug-attributable effects, and sequentially partitioning and assembling different sources of variation to accurately simulate the outcomes of a suitably normalized comparator group.

After decades of calls for greater data sharing [14-16] there are now many new platforms for accessing clinical trials data. The availability of these data has opened opportunities for researchers to verify published results as well as answer new questions using these data. This has never been more important, with the cost of new phase 3 clinical trials current at $20M and climbing [17].

Although the growing availability of IPD portends well for the future of research, it has revealed new analytical challenges that require new methods. Existing methods for conducting IPD meta-analyses typically involve including trials with near-identical study designs, including fully parallel-design cohorts and shared placebo comparator arms. When these criteria are not met, problematic trials are often excluded from a given meta-analysis, sometimes in subtle ways. This substantially limits the numbers of questions that might already be answerable using existing clinical data. In some cases, this common practice might even introduce bias.

The results above suggest there is a better way to handle this heterogeneity and discover new and trustworthy signals from these data. The method of the invention, as well as extensions therein, may substantially increase the numbers of studies that can be done, uncovering new evidence on comparative efficacy, safety, and ultimately precision medicine. Taking the example of Crohn's disease, a major motivation for conducting the SEAVUE trial is the current level of uncertainty regarding the comparative efficacy of already approved treatments. Methods such as what are propose here can address these gaps, particularly as more therapies are approved and thus the number of potential head-to-head comparisons grows exponentially.

While this methodology was illustrated in a comparative efficacy analysis, this approach may have significant value in other contexts. Models for the placebo effect, such as are demonstrates here, may help improve the design and statistical power of clinical trials across diseases. Moreover, the use of cohort normalization methods may be useful to improve the robustness of external control arm studies. These are studies that typically utilize real-world data to draw indirect inferences against controlled cohorts, typically single-arm intervention studies. Additionally, the above analysis suggests that a major driver of the large placebo effects in Crohn's disease is the large unmodeled variation in the CDAI. Accordingly, future work to improve the measurement of Crohn's disease activity may be warranted.

Several improvements may be made to enhance the above analysis. First, although extensive efforts were undertaken to harmonize the data, all covariate statistics could not be perfectly reproduce as published. It is likely that these issues could have been overcome with access to the original analytical code. Nonetheless, the degree of deviations from published results was small, and the primary results remained robust to many sensitivity analyses. Future efforts involving pre-harmonization to a common data model may improve the reproducibility and feasibility of these IPD meta-analyses. Second, many important covariates like race and ethnicity were unable to be included using the compiled studies. Most of the compiled studies did not capture ethnicity. Some studies did capture race but showed evidence of significant skew towards white participants. This likely reflects the historical under recognition of the importance of these factors. Lastly, it was noted that the validation was somewhat underpowered and was performed in the context of just one disease. This is largely a function of the relative rarity of clinical trials (the source of the present data and sample size), and especially head-to-head trials like SEAVUE. This underscores the importance of methods for learning more from these small but high-quality data. Future studies may be used to confirm the robustness and generalizability of the present invention methodology to other diseases.

In conclusion, a new method for meta-analyzing data from heterogeneous trials lacking a common control group was developed. This method was validated by reproducing the results of a recent comparative efficacy trial using pre-existing data.

### Example 2: Optimizing Treatment Selection in Crohn's Disease Using Patient-Specific Features

### Introduction

Multiple therapies with different mechanisms of action are now available for Crohn's disease (CD), a chronic and immune-mediated disorder of the gastrointestinal tract. These drugs have been approved on the basis of the overall superior outcomes of cohorts randomized to receive these treatments rather than placebo. What is less clear, however, is how to optimally select amongst them. Head-to-head trials are the gold-standard for comparing treatments, but they can be expensive and infeasible. Indeed, only one head-to-head trial of FDA-approved treatments for CD has been performed to date [10]. The barriers to high-quality evidence on comparative effectiveness and safety are further compounded by the growing number of treatments and the multiplicity of potential comparisons.

Thus, network meta-analyses (NMAs) have been the primary source of evidence on comparative efficacy/safety in CD. The most recent of these analyzed trials of three drug classes: anti-tumor necrosis factor alpha (anti-TNF), anti-integrin, and anti-interleukin-12/23 (anti-IL-12/23) [27]. The authors concluded that anti-TNFs were most effective at inducing remission, followed by anti-IL-23s.

While NMAs have provided important information about relative treatment effects, they are subject to many limitations. They assume that the included trials are homogenous across multiple dimensions (e.g., cohort risk profiles, study procedures, placebo effects). They also assume that the included trials represent a random sample of the potential comparisons of interest (e.g., an equal chance that an included trial will compare drug A to B rather than comparing drug A to placebo). They assume that the cohorts under study are an unbiased sample of real-world populations with active CD, a key assumption justifying the application of these results to practice. Lastly, they ignore the potential role of patient-level variation in explaining treatment outcomes. Although these models are able to infer relative treatment effects by averaging over the outcomes of a potentially heterogeneous population, they are less useful for identifying patient subgroups whose responses deviate from the majority.

The growing accessibility of raw data from clinical trials has created new opportunities to conduct individual participant data meta-analyses (IPDMAs). Although IPDMAs are more complicated to conduct, they offer greater analytical opportunities to mitigate unwanted heterogeneity across trials and to identify subgroups with different treatment responses. A method for normalizing the data from potentially heterogeneous clinical trials even in the absence of a consistent control group across studies was developed (see Example 1). This method, called sequential regression and simulation (SRS), was demonstrated in the context of nine randomized trials of treatment induction in CD and validated by using those data to successfully reproduce a major secondary outcome from the recently published SEAVUE trial. That trial found no cohort-averaged differences in effectiveness between adalimumab (anti-TNF) and ustekinumab (anti-IL-12/23).

Building on this framework, it was sought to test the hypothesis that clinically distinct disease subgroups with different treatment responses do exist and determine if more efficacious outcomes can be achieved by personalizing treatment selection rather than applying general rules (e.g., recommending anti-TNF as first line for all patients). Here, SRS was used to normalize the data from 15 trials (N=5703) corresponding to three major classes of FDA-approved drugs for Crohn's disease (anti-TNF, anti-integrin, anti-IL-12/23). The response to each drug class was then modelled as a function of patient-level characteristics, and classified patients into subgroups based on their treatment responses.

### Data Access

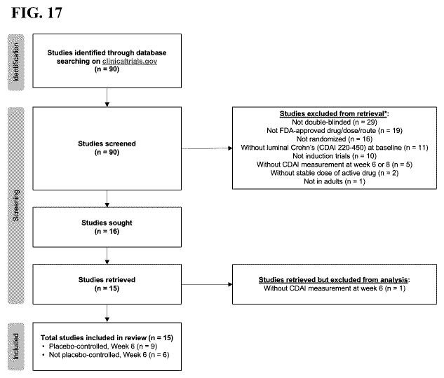

In June 2019, clinicaltrials.gov was queried to identify studies to include in this meta-analysis (FIG. 14A, FIG. 17). 16 trials were confirmed as being completed, phase 2-4, randomized, double-blinded, interventional trials of FDA-approved treatments for CD. These studies had similar cohort selection criteria and measured the Crohn's Disease Activity Index (CDAI) at week six after treatment initiation. Access to the IPD was successfully obtained for 15 studies (N=5703). These studies were conducted between 1999 and 2015 and corresponded to all six FDA-approved biologics as of 2019.

### Data Curation

Extensive quality control evaluations were performed of the obtained data. Nine variables were identified that were available across all trials and thus could be used for modelling: Age, Sex, BMI, baseline CDAI, c-reactive protein (CRP), history of TNFi use, oral steroid use, immunomodulator use, and ileal involvement.

Other important variables could not be included in this study. Ethnicity was not collected in most trials. Race was missing in some trials, but when it was captured, it reflected significant imbalance (88% of participants were white). Other disease specific variables such as disease behavior and duration were also not uniformly captured across studies.

The included trials had a range of study designs. both randomized and unblinded/open-label cohorts were included. For trials involving post-randomization gating (e.g., EXTEND, CLASSIC), those cohorts were included that were consistently exposed to a given treatment for six weeks only when post-randomization gating was not conditioned on treatment response.

### Drug Class Modelling, Subgroup Identification

Sequential regression and simulation (SRS) was used to 1) normalize all trials to a common background (placebo response), and 2) analytically isolate the portion of the patient response that could specifically be attributed to a given treatment, rather than what would have occurred without treatment (i.e. placebo; FIG. 14B) [29]. For each drug class, a separate linear mixed effects model was fit of the drug-attributable reduction in CDAI. This outcome was modelled as a function of the above nine primary variables as fixed effects, with trial as a random effect. These models were compared to intercept-only models using the likelihood ratio test. Intercept-only models ignore the role of patient-level characteristics in determining treatment responses and reflect the assumptions of methods that compare drugs based on their average effects (e.g., NMAs).

The three finalized models were applied to each of the 5703 participants to simulate their response under each of three counterfactual scenarios: treatment with an anti-TNF vs anti-integrin vs anti-IL-12/23. The inferred normal distributions of the conditional mean response to each drug class were pairwise compared against each other using the medians and standard errors of the bootstrapped predictions. A p=0.05 threshold was used to identify patients belonging to a particular subgroup, defined as having a distinct pattern of ordinal preferences across all three drug classes. These included superiority of one drug class over another, as well as approximate equivalence.

### Computing, Web Application

Programming was performed in R [30]. RShiny was used to prototype a decision support tool (crohnsrx.org). This web application utilizes manually inputted data to produce recommendations. For users seeking to develop and deploy this dashboard locally, an additional mode that automatically sources input data from an OMOP-formatted EHR database is available.

### Results

On Apr. 13, 2022, PubMed was queried to identify published studies that have used individual participant data from prospective studies to define a personalized treatment strategy in Crohn's disease. Search terms and filters included “Crohn's disease”, “individual participant data”, “meta-analysis”, and “precision medicine”. No candidate studies were identified. The query was revised to identify comparative efficacy meta-analyses using summary statistics from clinical trials. A recent and updated network meta-analysis evaluating comparative efficacy and safety was identified [27]. It found that anti-TNF drugs were generally the most effective at inducing clinical remission and thus should be considered as first-line treatment, followed by anti-IL-23 drugs as second line.

The study below, using the methods of the present invention, is the first, comprehensive, individual participant data (IPD) meta-analysis of comparative efficacy in Crohn's disease subgroups. The study incorporates many of the same randomized trials analyzed in the aforementioned meta-analysis, but specifically uses the raw data from these trials rather than published summary statistics. This approach is associated with two main advantages: 1) better control over many sources of cohort heterogeneity across trials, and thus a lower risk of bias, and 2) the ability to analyze subgroups.

The study identifies an alternative explanation for prior findings, that anti-TNF drugs are more efficacious than other drug classes, as actually being the result of a “majority vote” analysis. In fact most trial subjects (55%, N=3144) did not show strong evidence for a preference for any of the three drug classes studied here (anti-TNFs, anti-IL-12/23, anti-Integrin). Among the remaining subjects, more demonstrated superior efficacy with anti-TNFs compared to other drug classes (36%, N=2061).

Importantly though, evidence was also found of significant, implicit selection bias into these trials. A subgroup of women over 50 was identified who had superior efficacy with anti-IL-12/23s over other drug classes. While this group represented only 2% of the trial-based population, it was found that roughly 25% of the Crohn's patients who have been treated at University of California Health between 2012 and 2023 are women over 50. Of note, older age and female gender are not explicit exclusionary criteria for any of the trials meta-analyzed here. During the post-2016 period when all three drug classes were FDA-approved and available, it was found that 75% of biologic-exposed women over 50 did not receive an anti-IL-12/23 drug first line, suggesting potential room for treatment optimization.

To potentially aid future providers in utilizing these models to improve treatment selection, a treatment recommendation decision support tool was prototyped. This tool accepts manual input on patient-level features to recommend efficacious treatments based on the model. The tool can also be configured to automatically source patient-level data from EHR databases that conform to the OMOP data model and contain relevant inputs.

This study provides evidence that different patients with Crohn's disease have different inherent likelihoods of responding to different treatments, and that these proclivities can be predicted using commonly documented clinical features. Evidence of suboptimal treatment allocation was found in clinical practice and a decision support tool has been provided to potentially aid providers in selecting treatments. Given evidence for selection biases into registrational trials, decision support tools that account for patient-level features may enable a more precise application of clinical trial evidence to real-world patients, and potentially better outcomes. Trial designers should be cognizant of implicit selection biases and support efforts to improve the diversity and representativeness of these studies. A prospective study may be used to test the key predictions of this work and increase confidence in the underlying models prior to widespread application to clinical practice.

### Cohort Characteristics

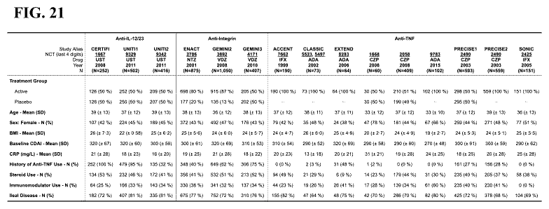

The cohort consisted of 5703 participants, drawn from fifteen trials of all FDA-approved biologics as of 2019. These biologics fell into one of three drug classes: anti-TNFs (infliximab, adalimumab, certolizumab), anti-IL-12/23s (ustekinumab), and anti-integrins (natalizumab, vedolizumab). The members of the cohort were generally similar by their univariate characteristics across trials (FIG. 21), although some heterogeneity was noted corresponding to treatment history and concomitant medications.

### Placebo Model

To address the potential bias that could result from a naive pooling of subjects across trials, sequential regression and simulation was used to normalize the data and analytically separate the drug-attributable component of the patient response from the drug-independent one (i.e. placebo effect) [29].

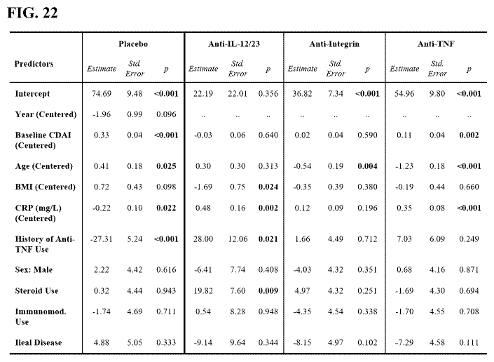

Using the subset of participants assigned to receive placebos (N=1621), their week 6 response was modelled as a function of all captured covariates and study year (fixed effects) as well as trial of origin (random effect). This model was highly significant (p<0.001; FIG. 22). Six predictors of the placebo effect were identified. The coefficient for study year was negative, suggesting a reduction in measured placebo effects over time and underscoring the importance of controlling for this source of inter-trial heterogeneity in meta-analyses. Male sex was associated with 27 points less of a placebo effect, as measured by CDAI reduction, compared to females. Baseline CDAI was also a significant predictor: every 100 points of a higher baseline CDAI (restricted by trial eligibility criteria to fall between 220-450) was associated with 33 points more of spontaneous improvement after 6 weeks. This was consistent with regression to the mean. Age and CRP were also significant albeit with small estimated effects. Most of the explainable variation in the placebo effect was accounted for by these explicitly captured clinical factors and study year; only 1% of the total variation was attributable to other non-specific heterogeneity across the included trials.

### Drug Class Models

The placebo model was used to calculate the mean placebo-attributable response for each participant who was assigned to receive active treatment (N=4082) and this was subtracted from their observed response, leaving behind the drug-attributable reduction in CDAI. The residuals were then used to fit three additional mixed effects models, one per drug class.

The drug class models were highly significant (p<0.01 for all; Table 2). 10 predictors were identified across drug classes. Efficacious responses to IL12/23s were positively associated with male sex (28 additional points of CDAI reduction) and steroid use (20 additional points). Elevated CRP was weakly associated with a positive response to IL12/23s, whereas elevated BMI was weakly associated with a negative response. For the anti-integrin class, each increased decade of life was associated with 5 points less of a response on the CDAI scale. Lastly, for the anti-TNF class 3 additional predictors of efficacy were identified beyond the intercept term. Elevations in baseline CDAI and CRP were associated with slightly increased efficacy, whereas age was inversely associated (12 points less of CDAI reduction for each additional decade).

To help improve the efficiency of future clinical trials, significant coefficients identified in the placebo and active treatment models were compared. Five coefficients had generally opposite effects across these two settings: age, BMI, CRP, male gender, and ileal involvement (FIG. 22). These results implied that young males with lower BMIs, elevated CRP and colonic disease would be expected to have the widest margin of difference between placebo and treatment arms, and thus the greatest power to detect evidence of efficacy. This finding also underscored the importance of separating placebo- and drug-attributable effects using separate regression models; a single regression model that lacks these implied interaction terms would miss these findings.

### Subgroup Analysis

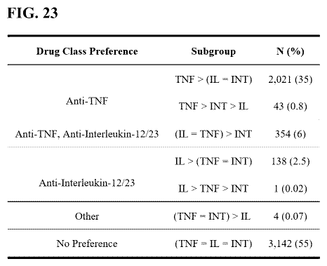

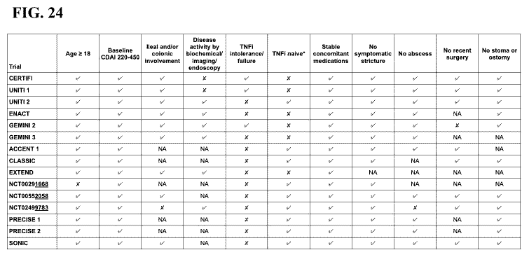

The potential outcomes of each participant with each drug class were simulated and pairwise t-tests were performed to rank-order their treatment preferences. These patterns of rank-ordering were used to classify participants into subgroups (FIG. 23). A majority of the trial participants (55%, N=3144) did not show strong evidence for superior efficacy with any one class over another. Most of the others showed evidence for superior efficacy with an anti-TNF (42%, N=2420). These results explain previously published findings favoring anti-TNFs as being the result of using ‘majority vote’ statistical methods in a situation where most participants ‘abstain.’

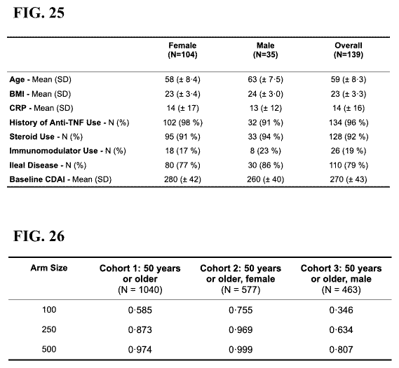

However, 139 participants showed evidence of superior efficacy with an anti-IL-12/23, achieving 40 points greater reduction on the CDAI compared to the other drug classes (FIG. 15; FIG. 25). 50% of these patients were predicted as achieving clinical response (CDAI reduction of 100 points or more) at week 6, compared to only 3% with an anti-TNF. This subgroup was predominantly female, over the age of 50, had a history of anti-TNF exposure, had relatively lower CDAIs at baseline, and were receiving steroids. This subgroup corresponded to only 2% of the trial population.

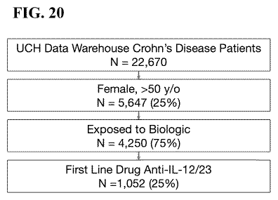

Given this, it was sought to determine whether a decision support tool might have any added value in clinical practice, compared to an easier-to-remember strategy of recommending an anti-TNF to any patient lacking a contraindication. The University of California Health Data Warehouse, a database of electronic health records data from 6 academic medical centers, was queried to identify potential patients who might belong to this subgroup and thus could benefit from a decision support tool making personalized recommendations. It was found that roughly 25% of the patients seen for Crohn's disease are women over the age of 50 (N=5,647) (FIG. 20). This striking difference in cohort prevalence (25% at the University of California vs 2% in the trials) suggested the possibility of implicit selection bias in these trials.

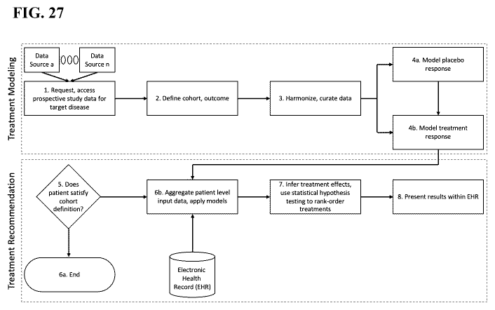

Since the existence of this anti-IL-12/23-preferring subgroup was a new and potentially testable hypothesis raised by this analysis, a sample size calculation was performed to determine the feasibility of verifying this in a prospective study (FIG. 26). A trial with 250 participants in each arm was calculated to have 87% power to show superiority of anti-IL-12/23s over anti-TNFs in all patients over the age of 50. If further restricted to just women over 50, this potential trial was calculated as having 97% power.

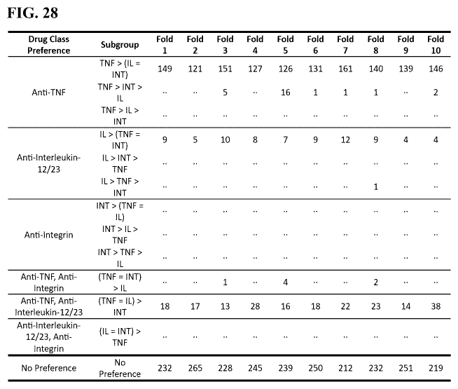

FIG. 28 depicts a table providing the results of a 10-fold cross-validation analysis demonstrating the robustness of treatment subgroup allocation using the linear mixed effect regression models of the study.

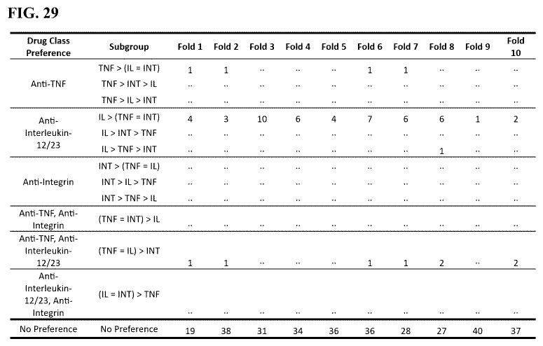

FIG. 29 depicts a table providing the results of a 10-fold cross-validation analysis demonstrating the consistent finding of an anti-interleukin-12/23 preference subgroup (female over 50) using the linear mixed effect regression models of the study.

### Decision Support Prototype

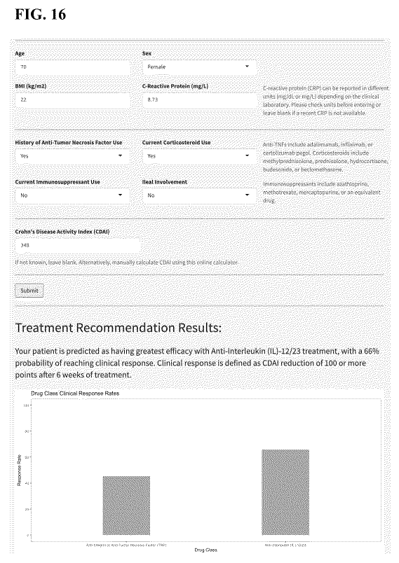

Finally, to evaluate the potential value of applying these findings to practice, a decision support tool was prototyped (crohnsrx.org). This tool uses manual inputs on patient-level features to produce treatment recommendations (FIG. 16). Additional guidance was provided to help clinicians interpret the output and avoid incorrectly using the tool on patients who do not resemble the subjects used to train the model.

### Methods

### Data Access

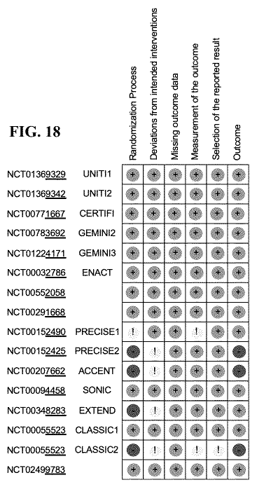

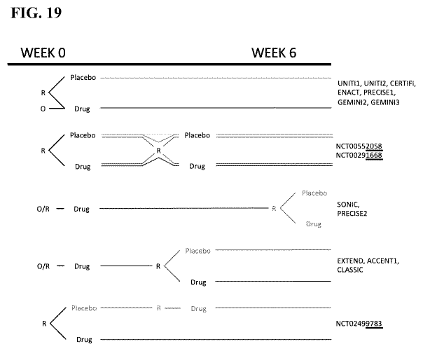

In June 2019 a search of clinicaltrials.gov was performed to identify candidate studies to include in this planned meta-analysis. 90 studies were identified that were annotated as being completed, phase 2-4, randomized, double-blinded, interventional trials of treatments for Crohn's disease at the FDA-approved route, dose, and frequency. 16 trials were manually confirmed as meeting these criteria. To ensure comparability of the included cohorts and outcomes, the major inclusion and exclusion criteria of all studies were reviewed and it was confirmed that the Crohn's Disease Activity Index (CDAI) had been captured at week six relative to treatment initiation. The Cochrane Risk of Bias 2 tool was also used to ensure that all included studies were at a low risk of bias (FIG. 18). Access to the IPD for 15 studies was successfully obtained (N=5703). These studies were conducted between 1999 and 2015 and corresponded to all six FDA-approved biologics as of 2019. All data was placed on a common, secure computing platform (Vivli) to facilitate downstream analysis.

### Quality Control, Harmonisation, Missing Data

Extensive quality control evaluations of the included trials and data were performed. This included confirming the ability to reproduce published statistics on the trial cohorts at baseline as well as the study primary endpoint. Most of the study results were able to be exactly reproduced. Where discrepancies occurred, they were generally minor and fell within a 10% error bound. Any potential major discrepancies were reported. It was attempted to completely eliminate all discrepancies, though this was not possible due to a variety of factors, including lack of access to the original analytic code or the complete analytic dataset, and inability to contact the original analysts.

An assessment of data availability was completed for all study variables. Target variables included demographic features, CDAI at baseline and week eight, baseline inflammatory biomarkers, concomitant steroid and immunomodulator use, history of treatment with anti-TNFs, and other disease-related features. Nine variables were identified that were universally available across all trials and thus could be used for downstream modelling: Age, Sex, BMI, baseline CDAI, c-reactive protein (CRP), history of TNFi use, oral steroid use, immunomodulator use, and ileal involvement.

Only 3% of the participants had at least one missing covariate at baseline. Continuous variables were addressed by median imputation, and participants with missing categorical variables were dropped from the dataset (N=86). 11% of the participants had a missing value for the outcome at week eight. To handle this, last-observation-carried-forward was used to impute these values, typically using measurements from week six and four. This is the typical practice for the analysis of these trials in regulatory submissions and was the prespecified approach in the protocols for all included trials. The variable corresponding to a history of TNFi use was available in all recent trials that occurred after the approval of the very first TNFi medication. Older trials of the first TNFis commonly excluded patients who had a history of exposure to other drugs from this class but did not include this feature as an actual variable in the data set. In these cases, this variable was deterministically imputed corresponding to no prior use.

Other variables of a priori importance could not be included in this study. Ethnicity was not collected in most trials. Race was missing in some trials, but when it was captured, it reflected significant imbalance (88% of participants were white). Other disease specific variables such as disease behavior and duration were also not uniformly captured across studies and thus could not be included in this meta-analysis.

The included trials had a range of study designs. Both randomized and unblinded/open-label cohorts were included. For trials involving post-randomization gating (e.g., EXTEND, CLASSIC), those cohorts were included that were consistently exposed to a given treatment for six weeks only when post-randomization gating was not conditioned on treatment response (e.g., rerandomization of all participants, rather than just those with a particular response).

### Drug Class Modelling, Subgroup Identification

Sequential regression and simulation was used to 1) normalize all trials to a common background (placebo response), and 2) analytically isolate the portion of the patient response that could specifically be attributed to a given treatment, rather than what would have been observed without treatment (i.e., placebo; FIG. 14B). For each drug class, a separate linear mixed effects regression model of the drug-attributable reduction in CDAI was fit. This outcome was modelled as a function of the nine primary variables (see the ‘Quality Control’ section above) handled as fixed effects, with trial as a random effect to control unmeasured heterogeneity across trials. These models were compared to intercept-only models using the likelihood ratio test. The latter corresponds to a model that ignores the role of patient-level characteristics in determining response to treatment and reflects the assumptions of methods that compare drugs based on their average effects, such as network meta-analyses. Wald tests were performed to identify significant predictors of responses to individual drug classes.

The three finalized model objects were applied to the covariate vectors of each of the 5703 participants in the meta-analysis to obtain their simulated response under each of these three counterfactual scenarios: treatment with an anti-TNF vs anti-integrin vs anti-IL-12/23. The inferred normal distributions of the conditional mean response to each drug class were pairwise compared against each other using the median of bootstrapped predictions and bootstrapped standard errors. A nominal p-value threshold of 0.05 was applied to identify patients belonging to a particular subgroup, defined as having a distinct pattern of ordinal preferences across all three drug classes. These included superiority of one drug class to another as well as indifference (lack of evidence for a difference at the p=0.05 threshold).

Because the primary focus of this study involved the testing of only three primary hypotheses (i.e., goodness of fit for each of the drug class regression models compared to intercept-only models), nominal p-value thresholds of 0.05 were used for all other hypothesis tests including the post-hoc assessments of drug subgroup membership.

### Subgroup Assignment

For each trial-based patient (N=5703) each drug class efficacy was predicted using the drug class models (FIG. 22; random effects set to 0) and estimated the 95% prediction interval using bootstrapping, the gold standard approach for deriving prediction uncertainty from linear mixed models [38]. 10,000 simulations were performed per patient. Paired sample t-tests (p<0.05) were conducted to further determine if any two drug class pairs were equivalent or different in efficacy to obtain a personalized treatment recommendation (FIG. 23). Finally, patients were assigned a subgroup based on their personalized treatment outcome based on the rank order and drug class comparisons.

### Decision Support Tool Prototype

The decision support tool has been developed to provide real-time feedback to clinicians selecting treatments for patients with moderate-to-severe Crohn's disease. However, a prototype of the tool is also being made publicly available to enable early feedback from many potential users and to provide insights to patients wishing to understand the potential advantages and disadvantages of available treatment options.

To use the decision support tool, users must input various data points, including the patient's age, gender, body mass index (BMI), recent c-reactive protein levels (measured in milligrams per liter), current corticosteroid and immunomodulator use (yes/no), prior anti-tumor necrosis factor use (yes/no), ileal involvement (yes/no), and the Crohn's Disease Activity Index (CDAI) score. All inputs, except for the CDAI score, are mandatory for the calculation process. If any inputs are left blank, the user will receive an error message (FIG. 16) and be prompted to input a default of ‘0’ for numeric inputs or ‘No’ for binary inputs if unknown. If the CDAI score is unknown, the user can either 1) leave it blank, which will result in the tool imputing a score of 300 (indicative of moderate-to-severe disease), or 2) use the MDCalc CDAI calculator to obtain a precise result.

If all inputs are valid, the dashboard will output the patient's treatment recommendations in both textual and graphical forms (FIG. 16). To achieve faster recommendations in a real-time context compared to what would otherwise be obtained using bootstrapping, an analytical approximation was used for the standard error of a new prediction [40]. These standard errors were used to perform t-tests of the predicted mean response at week 6 for each pair of drug classes.

### University of California Health Data Warehouse

The University of California (UC) Health Data Warehouse (UCHDW) contains data on 8.7 million patients who have been seen at a UC facility since 2012; data has been stored using the Observational Medical Outcomes Partnership (OMOP) data model. Additional information about the OMOP common data model can be found at (www.ohdsi.org).

The UCHDW was queried to approximate a real-world subpopulation with similar characteristics to that of the anti-IL-12/23 subgroup found in the analysis, which consists of primarily older (>50 years old) and female participants. Queries were run on Apr. 5, 2023. Patients were filtered in the UCHDW based on diagnoses (Crohn's disease), medication prescriptions (adalimumab, ustekinumab, infliximab, natalizumab, vedolizumab, certolizumab pegol), medication start date, current age (as of 2023), and gender (FIG. 20). Standard concept ids for diagnoses and medications were identified using the SNOMED International SNOMED CT Browser, Athena [17]. The codes are listed here: Crohn's disease (201606), adalimumab (1119119), ustekinumab (40161532), infliximab (937368), natalizumab (735843), vedolizumab (45774639), and certolizumab pegol (912263). For more details on the query, the code may be found on (github.com).

### Sample Size Calculations

Simulations were performed to calculate the expected power of a prospective trial designed to test a key prediction of the model, that anti-IL-12/23 drugs are superior to anti-TNF drugs in women over 50. In each of 1000 simulations, the overall trial population was sampled from to create pairs of study arms consisting of women over 50. Sampling was done with replacement. The placebo and drug-models were used to calculate the individual-level probability of achieving a CDAI reduction of ≥100 (i.e., clinical response), under an assumption of conditional normality. These were averaged within each simulated study arm, used to calculate the expected number of participants in clinical response, and then compared using a chi-squared test with an alpha of 0.05. This overall simulation procedure was performed using study arm pairs of sizes 100, 250, and 500. This analysis was repeated with the simpler inclusion criteria of just requiring participants to be over age 50, irrespective of gender.

### Statistical Computing, Web Application Development

Programming was performed in R (version 4.2.2). RShiny was used to prototype a decision support tool implementing the created models (crohnsrx.org). The analytical code was reviewed by multiple individuals and has been placed on GitHub (github.com). The web dashboard utilizes manually-inputted data to produce recommendations based on the models. However, for users seeking to deploy this dashboard locally, an additional mode may be used that automatically sources input data from an OMOP-formatted EHR database.

### SUMMARY

A subgroup analysis was performed of individual participant data (IPD) from 15 trials of treatments for Crohn's disease. The primary findings can be summarized as follows: 1) patients with Crohn's disease likely harbor different underlying preferences towards different treatments, and these preferences are partially predictable using clinical features, 2) most trial participants do not appear to have superior efficacy with anti-TNFs drugs, a potentially unexpected finding given prior literature, 3) there appears to be evidence of significant implicit selection bias into registrational trials, and 4) the use of statistically-based decision support tools may improve patient outcomes in clinical practice. Secondary results include 1) newly-identified features that predict patient-level responses to different drugs as well as placebo, and insights as to how they could be used to design more efficient clinical trials, and 2) sample size calculations supporting the feasibility of testing this model's predictions prospectively. Overall, these findings add to a growing body of evidence that shifts away from a ‘one-size-fits-all’ treatment paradigm and towards one of precision medicine.

The work directly builds on previous evidence synthesis efforts in Crohn's disease, particularly network meta-analyses (NMAs) that compared treatment efficacy and safety [27, 32]. The most recent of these found that anti-TNF drugs appear to be the most efficacious drug for inducing clinical remission, and that they should generally be recommended as first line agents over other drug classes like anti-IL-12/23s [27]. Although a very similar set of trials was used as that study, a different conclusion was reached: most of the subjects in these trials do not appear to preferentially benefit from any of three currently approved drug classes. Instead, it was found that patients favoring anti-TNFs were actually in the minority, albeit a large one (42%). This apparent contradiction can be understood as the result of an “ecological fallacy”, where one incorrectly deduces that a cohort-averaged effect also applies to each member of the cohort. An apt analogy would be of an election where the majority abstains, and the next largest constituency ‘votes’ for an anti-TNF.

Thus, the findings of the created models are in fact consistent with prior NMAs that instead rely on aggregate statistics from trials. However, these findings also further suggest that the field of evidence synthesis must increasingly embrace IPD to generate results that are more accurate and less susceptible to misinterpretation. Methods such as sequential regression and simulation can add additional credibility and reduce the dependence on strong homogeneity assumptions implicit in traditional pooled analyses of IPD. This method also enables deeper insights into the overall patient response as the result of two analytically distinguishable effects: placebo-attributable and drug-attributable. The predictors of the placebo effect that were identified here were consistent with the prior literature [33]. Yet more predictors were identified than have previously been reported, likely because the above method of the present invention implicitly accounts for drug-by-effect interactions that are often not modelled in one-step IPD meta-analyses [28, 2]. The value of these findings, beyond that of scientific interest in how these clinical features reflect treatment mechanisms and disease biology, is also practical. The results suggest concrete ways that clinical trials could be designed to be more efficient, i.e., yield greater expected differences between placebo and treatment arms.

Another important but unexpected finding from this work is evidence of potentially significant implicit selection bias into Crohn's RCTs. Some degree of selection bias is to be expected of all trials insofar as they contain additional inclusion and exclusion criteria that are not a requirement for receiving clinical care. Indeed, this study has observed this in the context of direct comparisons between RCT cohorts and their real-world counterparts [34-36]. What has been less clear, however, is the extent to which these biases may distort treatment outcomes. A subgroup of anti-IL-12/23-preferring patients was identified, mostly women over 50, that represented a miniscule fraction of trial subjects (2%). Yet, the typical prevalence of these patients as seen across 6 medical centers at the University of California suggests that as many as 25% patients fall into this demographic. Of course, gender and older age are not explicit exclusionary criteria in these trials, most of which were submitted to the FDA prior to new drug approvals. Thus, it appears that these patients are systematically being under-enrolled. Future studies are needed to determine if this is the result of patient preferences, provider biases, or other factors.

Artificial intelligence is playing a growing role in healthcare, with a proliferation of data-informed software tools that can help clinicians make more informed decisions. Yet, many uncertainties remain as to how these models should be rigorously tested and deployed in clinical practice [37]. Clinical decision support tools need rigorous testing in typical practice settings before they can be trusted and widely deployed. Prospectively testing the described model in its entirety is generally a challenge because it can make predictions across the continuum of patient features. Many patients did not appear to have a strong treatment preference, making it challenging to envision a study that ‘confirms’ this null hypothesis. By contrast, the aforementioned subgroup of older women is ideal from a prospective standpoint: 1) it is scientifically new and undescribed in the literature, 2) it demonstrates a treatment effect that can be evaluated via null hypothesis testing, and 3) its members are sufficiently prevalent in real-world settings, increasing the potential feasibility of subject recruitment.

Separate from testing the statistical model is testing the decision support prototype itself. An early version (crohnsrx.org) has been tested to facilitate early user feedback and enhancement. This model may be developed into an EHR-embedded tool that supports seamless, timely, and trustworthy recommendations at the point of care.

Strengths of this work include the strength and quality of the underlying data, the use of the SRS method to address bias and reveal new insights, as well as many important findings impacting the science, study, and care of patients with Crohn's disease.

Several improvements may be made to enhance the above analysis. This was a post-hoc analysis of randomized trials, and therefore the possibility of residual bias cannot be completely excluded. Prospective studies may be used to test these findings, particularly given apparent selection biases that could degrade the application of trial-based insights to clinical practice. There were several variables that could be included in the models using the compiled trials such as race and smoking as, unfortunately, these data were not well-captured across the included compiled trials. A large amount of unexplained variability in patient outcomes were found, roughly 90%. This is not a weakness of the study, but rather reflects the large scope of future work that is needed to explain patient outcomes in Crohn's disease. Some of this work will benefit from the discovery of new biomarkers (e.g., metagenomic, metabolomic), none of which were available during creation of the described models, though they may seamlessly be incorporated into future iterations. Other work increasing the objectivity of disease activity measures in Crohn's disease and reduce unwanted variation may be beneficial. Lastly, possible information leakage from training each model on a subset of the cohort's data was acknowledged. To mitigate bias, the median of bootstrapped predictions and standard errors was used to estimate the distribution for each patient's response.

In conclusion, an IPD meta-analysis of RCTs in Crohn's disease was performed. Multiple subgroups were identified with different preferential responses to different drug classes, including one subgroup of women over 50 who may respond favorably to anti-IL-12/23s. Potential evidence of selection bias in clinical trials was uncovered and ways to improve the efficiency and equity of these gold-standard studies was suggested. Lastly, a prototype decision support tool was developed that implements these findings that may be used to help improve treatment selection and patient outcomes in Crohn's disease.

### REFERENCES

[1] Chalmers I. The Cochrane collaboration: preparing, maintaining, and disseminating systematic reviews of the effects of health care. Ann N Y Acad Sci 1993; 703:156-63; discussion 163-5.
[2] Tierney J F, Vale C, Riley R, et al. Individual Participant Data (IPD) Meta-analyses of Randomised Controlled Trials: Guidance on Their Use. PLOS Med 2015; 12: e1001855.
[3] Schmitz S, Adams R, Walsh C. Incorporating data from various trial designs into a mixed treatment comparison model. Stat Med 2013; 32:2935-49.
[4] Ibrahim J G, Chen M H. Power Prior Distributions for Regression Models. Stat Sci 2000; 15:46-60.
[5] Zhang J, Ko C W, Nie L, Chen Y, Tiwari R. Bayesian hierarchical methods for meta-analysis combining randomized-controlled and single-arm studies. Stat Methods Med Res 2019; 28:1293-310.
[6] Thompson L, Chu J, Xu J, Li X, Nair R, Tiwari R. Dynamic borrowing from a single prior data source using the conditional power prior. J Biopharm Stat 2021; 31:403-24.
[7] Wang C, Li H, Chen W C, et al. Propensity score-integrated power prior approach for incorporating real-world evidence in single-arm clinical studies. J Biopharm Stat 2019; 29:731-48.
[8] Li X, Song Y. Target Population Statistical Inference With Data Integration Across Multiple Sources—An Approach to Mitigate Information Shortage in Rare Disease Clinical Trials. Stat Biopharm Res 2020; 12:322-33.
[9] Li X, Miao W, Lu F, Zhou X. Improving efficiency of inference in clinical trials with external control data. Biometrics 2021; biom.13583.
[10] Sands B E, Irving P M, Hoops T, et al. Ustekinumab versus adalimumab for induction and maintenance therapy in biologic-naive patients with moderately to severely active Crohn's disease: a multicentre, randomised, double-blind, parallel-group, phase 3b trial. Lancet Lond Engl 2022; 399:2200-11.
[11] PROSPERO Comparative effectiveness of treatments for moderate-to-severe Crohn's disease: an individual participant data meta-analysis. (accessed Sep. 28, 2021).
[12] The YODA Project 2018-3476. Effic. Crohn's Dis. Treat. Stratif. Dis. Phenotype. (accessed Sep. 28, 2021).
[13] Efficacy of Crohn's Disease Treatment Stratified by Disease Phenotype. Vivli. (accessed Sep. 28, 2021).
[14] Mello M M, Francer J K, Wilenzick M, Teden P, Bierer B E, Barnes M. Preparing for Responsible Sharing of Clinical Trial Data. N Engl J Med 2013; 369:1651-8.
[15] Packer M. Data sharing in medical research. BMJ 2018; 360: k510.
[16] Health Data Sharing Special Publication. Natl. Acad. Med. (accessed Aug. 10, 2022).
[17] Examination of Clinical Trial Costs and Barriers for Drug Development. ASPE. (accessed Jun. 12, 2022).
[18] Wickham H, François R, Henry L, Müller K. dplyr: A Grammar of Data Manipulation. Published online 2022.
[19] Bates D, Mächler M, Bolker B, Walker S. Fitting Linear Mixed-Effects Models Using lme4. J Stat Softw. 2015; 67:1-48. doi: 10.18637/jss.v067.i01
[20] Kuznetsova A, Brockhoff P B, Christensen R H B. ImerTest Package: Tests in Linear Mixed Effects Models. J Stat Softw. 2017; 82:1-26. doi: 10.18637/jss.v082.i13
[21] Dowle M, Srinivasan A, Gorecki J, et al. data.table: Extension of “data.frame.” Published online Sep. 27, 2021. Accessed Aug. 29, 2022.
[22] Wickham H. Ggplot2: Elegant Graphics for Data Analysis. Springer; 2016.
[23] Kassambara A. ggpubr: “ggplot2” Based Publication Ready Plots. Published online Jun. 27, 2020. Accessed Aug. 29, 2022.
[24] Lüdecke D. sjstats: Collection of Convenient Functions for Common Statistical Computations. Published online Jan. 9, 2021. Accessed Aug. 29, 2022.
[25] Pedersen T L. patchwork: The Composer of Plots. Published online Aug. 19, 2022. Accessed Aug. 29, 2022.
[26] Auguie B, Antonov A. gridExtra: Miscellaneous Functions for “Grid” Graphics. Published online Sep. 9, 2017. Accessed Aug. 29, 2022.
[27] Singh S, Murad M H, Fumery M, et al. Comparative efficacy and safety of biologic therapies for moderate-to-severe Crohn's disease: a systematic review and network meta-analysis. Lancet Gastroenterol Hepatol 2021; 6:1002-14.
[28] Stewart L A, Tierney J F. To IPD or not to IPD? Advantages and disadvantages of systematic reviews using individual patient data. Eval Health Prof 2002; 25:76-97.
[29] Rudrapatna V A, Ravindranath V G, Arneson D V, Mosenia A, Butte A J, Wang S. A method for estimating causal effects from heterogeneous clinical trials without a common control group using sequential regression and simulation. 2022; 2022.10.07.22280801.
[30] R Core Team. R: A language and environment for statistical computing. 2023. R-project.org.
[31] Chang W, Cheng J, Allaire J J, et al. shiny: Web Application Framework for R. 2023 shiny.rstudio.com.
[32] Cholapranee A, Hazlewood G S, Kaplan G G, Peyrin-Biroulet L, Ananthakrishnan A N. Systematic review with meta-analysis: comparative efficacy of biologics for induction and maintenance of mucosal healing in Crohn's disease and ulcerative colitis controlled trials. Aliment Pharmacol Ther 2017; 45:1291-302.
[33] Systematic review with meta-analysis: placebo rates in induction and maintenance trials of Crohn's disease—Jairath—2017—Alimentary Pharmacology & Therapeutics—Wiley Online Library. (accessed May 10, 2023).
[34] Ha C, Ullman T A, Siegel C A, Kornbluth A. Patients enrolled in randomized controlled trials do not represent the inflammatory bowel disease patient population. Clin Gastroenterol Hepatol Off Clin Pract J Am Gastroenterol Assoc 2012; 10:1002-7; quiz e78.
[35] Rudrapatna V A, Glicksberg B S, Butte A J. Utility of routinely collected electronic health records data to support effectiveness evaluations in inflammatory bowel disease: a pilot study of tofacitinib. BMJ Health Care Inform 2021; 28: e100337.
[36] Rudrapatna V A, Cheng Y W, Feuille C, et al. Creation of an ustekinumab external control arm for Crohn's disease using electronic health records data: A pilot study. PloS One 2023; 18: e0282267.
[37] Center for Devices and Radiological Health. Clinical Decision Support Software. U S Food Drug Adm. 2022; published online September 22. [fda.gov] (accessed May 10, 2023).
[38] Knowles J E, Frederick C. merTools: Tools for Analyzing Mixed Effect Regression Models. 2023.
[39] Crohn's Disease Activity Index (CDAI). MDCalc. [mdcalc.com] (accessed May 10, 2023).
[40] Francq B G, Lin D, Hoyer W. Confidence, prediction, and tolerance in linear mixed models. Stat Med 2019; 38:5603-22.
[41] Athena. [athena.ohdsi.org] (accessed May 10, 2023).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group. As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims. The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

1. A method of modeling an effect of a placebo on a subject having a disease or condition, the method comprising:
obtaining individual participant data (IPD) from a plurality of clinical trials regarding the treatment of the disease or condition with a medical intervention;
processing a subset of the obtained IPD including only data associated with individuals that have received a placebo, wherein the processed IPD includes one or more patient level features and an outcome variable for each individual; and
fitting a mixed effects regression model to the processed subset of IPD, wherein the one or more patient level features of each individual and the clinical trial associated with each individual are modeled as covariates.

2. The method according to claim 1, wherein the plurality of clinical trials lack a shared control group.

3. The method according to claim 1, wherein the one or more patient level features of each individual are modeled as fixed effects, wherein the clinical trial associated with each individual is modeled as a random effect.

4. (canceled)

5. The method according to claim 1, wherein the period in time when each clinical trial was performed is modeled as a covariate.

6. (canceled)

7. The method according to claim 1, wherein the subset of the IPD includes all individuals that have received a placebo or a random sampling of individuals that have received a placebo.

8-10. (canceled)

11. The method according to claim 1, wherein the disease or condition is a chronic disease or condition.

12. The method according to claim 1, wherein the one or more patient level features are selected based on the number of individuals of the obtained IPD for which the one or more patient level features are included,
wherein the one or more patient level features are selected based on the disease or condition and/or the medical intervention, or
wherein one or more of the patient level features is associated demographic information, chronic disease burden, a sign or symptom of the disease or condition, and/or a previously known modifier of response to the medical intervention.

13-15. (canceled)

16. The method according to claim 1, wherein the processing comprises filtering and/or harmonizing the subset of obtained IPD.

17. The method according to claim 16, wherein the subset of obtained IPD is filtered to include only data generated from clinical trials that are completed, phase 2-4, randomized, double blind, interventional, and/or approved by the U.S. Food and Drug Administration (FDA);
wherein the subset of obtained IPD is filtered to include only data generated from clinical trials including a minimum threshold of continuous observation time of one or more subjects on a placebo relative to a baseline; or
wherein the subset of obtained IPD is filtered to exclude participants for which one or more of the patient level features are not included, wherein the patient level feature not included is a categorical variable.

18-21. (canceled)

22. The method according to claim 16, wherein the harmonizing comprises imputing one or more of the patient level features and/or the outcome variable for a participant of the obtained IPD missing the one or more patient level features and/or the outcome variable.

23. The method according to claim 22, wherein the imputing comprises median imputation of when the patient level feature and/or the outcome variable is a continuous variable,
wherein the imputing comprises a last observation carried forward (LOCF) imputation, or
wherein the imputing comprises replacing the missing patient level feature using domain specific knowledge.

24-25. (canceled)

26. The method according to claim 16, wherein the filtering comprises performing a quality control evaluation.

27. The method according to claim 26, wherein the quality control evaluation comprises generating a quality metric for each clinical trial.

28. The method according to claim 27, wherein the obtained IPD is filtered to exclude participants associated with clinical trials having a generated quality metric below a predetermined threshold value.

29. (canceled)

30. The method according to claim 1, wherein the mixed effects regression model comprises a statistical regression model and/or a machine learning (ML) model;
wherein the mixed effects regression model comprises a statistical regression model;
wherein the mixed effects regression model comprises a linear mixed effects regression mode; or
wherein the mixed effects regression model comprises a supervised ML model.

31-35. (canceled)

36. The method according to claim 1, wherein the disease or condition is a polygenetic disease, Crohn's disease, a hereditary disease, acute hepatic porphyria, primary hyperoxaluria, or hereditary transthyretin amyloidosis.

37. (canceled)

38. The method according to claim 1, wherein the medical intervention comprises i) anti-TNF, anti-integrins, or anti-IL-12/23, or ii) a pharmaceutical composition, a medical device, a surgery, and/or a therapy.

39-44. (canceled)

45. The method according to claim 1, further comprising designing a clinical trial using the fitted regression model.

46. The method according to claim 1, further comprising identifying a relationship between the outcome variable and one or more of the patient specific features using the fitted regression model.

47. The method according to claim 46, wherein one or more of the patient specific features is identified as a positive predictor of the placebo effect or a negative predictor of the placebo effect.

48. (canceled)

49. The method according to claim 1, further comprising processing a second subset of the IPD including only data associated with individuals that have received a first medical intervention.

50. The method according to claim 49, further comprising determining a component of the outcome variable attributable to the first medical intervention for each individual of the second subset using the one or more patient level features of each individual and the fitted regression model.

51. The method according to claim 50, further comprising fitting a medical intervention regression model to the first medical intervention attributable component and the one or more patient level features of each individual of the second subset,
wherein the fitted medical intervention regression model is used to identify relationships between the first medical intervention attributable component and the one or more patient level features, or
wherein one or more of the patient specific features is identified as a positive predictor of patient response to the first medical intervention or a negative predictor of patient response to the first medical intervention.

52-54. (canceled)

55. A method of modeling an effect of a medical intervention on a subject having a disease or condition, the method comprising:
obtaining individual participant data (IPD) from a plurality of clinical trials regarding the treatment of the disease or condition with one or more medical interventions, wherein the clinical trials lack a shared control group;
processing a first subset of the obtained IPD including only data associated with individuals that have received a placebo and a second subset of the obtained IPD including only data associated with individuals that have received a first medical intervention, wherein the processed IPD includes one or more patient level features and an outcome variable for each individual; and
fitting a placebo regression model to the processed first subset of IPD in order to determine an effect of each patient level feature on the outcome variable;
determining a component of the outcome variable attributable to the first medical intervention for each individual of the processed second subset using the one or more patient level features of each individual and the fitted placebo regression model;
fitting a first medical intervention regression model to the first medical intervention attributable component and the one or more patient level features of each individual of the second subset, wherein the one or more patient level features of each individual of the second subset are modeled as covariates.

56-110. (canceled)

111. A method of generating a personalized treatment recommendation for a subject having a disease or condition and one or more patient level features, the method comprising:
obtaining individual participant data (IPD) from a plurality of clinical trials regarding the treatment of the disease or condition with multiple different medical interventions, wherein the clinical trials lack a shared control group;
processing a subset of the obtained IPD including only data associated with individuals that have received a placebo and a subset of the obtained IPD for each of the multiple different medical interventions including only data associated with individuals that have received a specific medical intervention of the multiple different medical interventions, wherein the processed IPD includes the one or more patient level features and an outcome variable for each individual; and
fitting a placebo regression model to the processed placebo subset of IPD in order to determine an effect of each patient level feature on the outcome variable;
determining, for each medical intervention subset, a component of the outcome variable attributable to the specific medical intervention of the respective intervention subset for each individual of the subset using the one or more patient level features of each individual and the fitted placebo regression model;
fitting, for each medical intervention subset, a medical intervention regression model to the medical intervention attributable component and the one or more patient level features of each individual of the respective intervention subset, wherein the one or more patient level features of each individual of the intervention subset are modeled as covariates; and
generating the personalized treatment recommendation for the subject using each medical intervention regression model and the one or more patient level features of the subject.

112-126. (canceled)
